(19) United States
(12) Patent Application Publication
(10) Pub. No.: US 2010/0273258 A1
(43) Pub. Date: Oct. 28, 2010

(54) Interactive Microenvironment System

(75) Inventors: John J. Lannutti, Grove City, OH (US); Jed K. Johnson, Columbus, OH (US); E. Antonio Chiocca, Powell, OH (US); Sara Nicole Fischer, Columbus, OH (US); Sean E. Lawler, Columbus, OH (US); Young C. Lin, Worthington, OH (US); Clay B. Marsh, Columbus, OH (US)

Correspondence Address: MUELLER AND SMITH, LPA;MUELLER-SMITH BUILDING, 7700 RIVERS EDGE DRIVE, COLUMBUS, OH, 43235, US

(73) Assignee: The Ohio State University, Columbus, OH (US)

(21) Appl. No.: 12/766,328

(22) Filed: Apr. 23, 2010

Related U.S. Application Data
(60) Provisional application No. 61/172,294, filed on Apr. 24, 2009.
(60) Provisional application No. 61/182,948, filed on Jun. 1, 2009.

(51) Int. Cl. C12N 5/071; C12M 3/00

(52) U.S. Cl. 435/366; 435/289.1

(57) ABSTRACT

A culture cell for growing animal cells in vitro has sides and a bottom forming a volume. The volume contains a layer of nanofiber upon which animal cells can be cultured. The layer of nanofiber can be oriented or non-oriented. Multiple layers can be placed in the volume, where the layers have different composition and/or different porosity. The nanofiber can be, for example, surface treated or of a core-shell construction.

### CROSS-REFERENCE TO RELATED APPLICATIONS

This application claims priority to U.S. provisional patent applications Ser. Nos. 61/172,294 filed on Apr. 24, 2009; and 61/182,948 filed on Jun. 1, 2009, the disclosures of which are expressly incorporated herein by reference.

### STATEMENT REGARDING FEDERALLY SPONSORED RESEARCH

The data reported herein was sponsored by National Science Foundation under Grant No. EEC-0425626.

### BACKGROUND

The present disclosure relates to multi-culture microenvironment systems and more particularly to a cell culture microenvironment system utilizing electrospun fiber, which may be oriented and multi-layered.

One limitation in cell culture for tissue engineering and other biomedical applications has been the poor resemblance to the topographical richness of the in vivo environment. Tumors develop and progress in complex three-dimensional microenvironments in which both normal and cancerous cells encounter specific physical, chemical, and biological challenges. Following these exposures, some cancer cells display uncontrolled growth, invasiveness and the ability to metastasize, often along aligned biological structures. Others undergo apoptosis and disappear. These challenges constitute a critical event defining the eventual prognosis of patients who present with cancer. Because the interactions between cells and the dietary components present in their microenvironments are dynamic, there has been intense interest in identifying mechanisms by which cells are influenced by these components. However, the role dietary influences play in the dynamic interplay between tumor cells and the physical and chemical challenges around them is uncertain. Part of the reason is that in vivo systems, and even complex tumor-derived matrices, do not allow for deconstruction of these parameters in a reproducible and rigorous manner.

Conventional 2-D culture distorts tumor cell activity because planar systems are unable to faithfully recapitulate in vivo cell interactions with the surrounding microenvironment. In comparison to their ‘normal’ in vivo state, on tissue culture polystyrene (TCPS) cell morphology, metabolism, gene and protein expression, differentiation patterns, and intracellular signaling are greatly altered. In all mammalian tissues, cells exist within a network of ECM made up of a randomly organized mesh of small, highly compliant fibers ˜0.3 to 3 μm in diameter. In contrast to the behavior often observed on TCPS, these ECM-based fibers usually are compliant enough that they do not provide anchorage points for the formation of intracellular stress fibers in motile cells. Thus, the microenvironments, in which tumorigenesis takes place, are fibrillar and compliant in nature. In addition, cancer cell invasion and metastasis involve traversing fibrillar/submicron fibril structures that provide both an appropriate physical resistance and molecular anchors that permit the formation of focal adhesions and thus cell motility. In addition, the vast majority of experiments involving TCPS use only monocultures involving a specific cell line. The use of conditioned cell media shows considerable promise (1-4) although a lack of standardization, particularly in regards to media age, can make interstudy comparisons difficult.

Electrospun fiber has been widely used as a tissue-engineering scaffold. The resemblance of electrospun fibers to the natural extra-cellular matrix that surrounds mammalian cells in vivo has spawned applications in the reconstruction of human tissue around the world. Standard cell culture on standard electrospun fiber constitutes an important improvement over tissue culture polystyrene, but usually involves only one type of cell.

Unfortunately, prior electrospun fiber does not mimic in vivo cells and cell layers sufficiently to provide the researcher an in vitro model of an in vivo cellular environment. It is an unmet advantage, then, of the prior art to utilize electrospun fibers to provide a more physiologically and topographically similar microenvironment culture system that can maintain, for example, viability of multiple cell types in various desired arrangements.

### BRIEF SUMMARY

A culture cell for growing animal cells in vitro, has sides and a bottom forming a volume. The volume contains a layer of nanofiber upon which animal cells can be cultured. The layer of nanofiber can be oriented or non-oriented. Multiple layers can be placed in the volume, where the layers have different composition or different porosity. The nanofiber can be surface treated or of a core-shell construction.

Uses of the disclosed nanofiber for cell culturing are varied. Some of such uses include, inter alia, cells can be ‘sorted’ or separated from one another on the basis of motility. This will allow subsequent analysis, for example of genetic differences allowing the development of specific medical treatments. Cutting of nanofiber using a laser or any other energy source allows control over the direction of cell motion. Manipulating or arranging the fibers through magnetic or mechanical means such as AFM tips may produce valuable properties.

Integration of aligned fiber arrays with any other cell manipulation device for further cell processing or separation, such as, for example, fluorescence-activated cell sorting (FACS) or a magnetic trap array or optical tweezers. Adaptation of electrospun fiber to a multi-layer co-culture system involving, for example, Transwell® inserts or any other insert allowing multicellular communication between cell populations; creation of multi-layer versions in which fibers of different porosity (interfiber spacing) host cells that can enable multicellular communication with each other; aligned fiber as a mimic of white matter, blood vessels, milk ducts or any other biological tissue consisting of aligned nanofiber or nanofibrils; and aligned or unaligned versions of these nanofibers.

The disclosed nanofiber layers can create in vivo microenvironments via chemical means involving the ‘signaling’ generated by the addition of neighboring cells. Addition of post-spinning bioactivity via applied coatings (e.g., via inkjet printing or the like) or super-critical or sub-critical CO₂ treatments to these fibers helps to create valuable biological behavior. Addition of surface treatments to the fibers such as, for example, superhydrophobicity or superhydrophilicity may prove valuable. Addition of a chemotactic source to these fibers helps to guide the cells in a specific direction, usually parallel to the fiber axis. This source may be applied utilizing inkjet printing.

Conditioning the fibers with culture media and/or cells to add bioactivity can be practiced. The use of any high volatility solvent, such as, for example, hexafluoroisoproponal (HFIP or HFP), acetone, dichloromethane, trifluoroacetic acid, acetic acid, petroleum either, dimethylformamide and others aids in the electrospinning of the aligned fiber arrays. Alignment using a rotating ground or a “split ground” deposition and/or electrostatic focusing methods creates improvements in alignment.

Different moduli polymer fibers, such as, for example, polycaprolactone, polyethersulfone, or polyethylene terephthalate that may influence biological behavior can be aligned utilizing methods identical to those described earlier. Different polymer blends or core/shell structures to achieve different mechanical properties or biological activity can be practice, such as, for examople, blending polycaprolactone and gelatin to increase bioactivity.

Fiber diameters can range from about 5 nanometers to as much as about 50 microns. Surface morphologies (pores, dimples, grass or hair shapes) can be created using techniques, for example, core-shell electrospinning, atmospheric additions/changes during electropsining, plasma etching in various atmospheres and others.

The application of this technique to multi-well production produces a range of products varying from simple large petri dishes to 1536-well plates and including multi-well slide chambers.

Variable linear fiber densities ranging from about 1 fiber/mm to about 200,000 fibers/mm can be useful in producing valuable improvements in cell behavior. Uses in the in vitro evaluation of cosmetic products, various forms of radiation exposure, chemotherapeutics and other cancer-related chemical compounds, dietary influences on cell development, cell-cell communication, anti-migratory compounds, cell separations, oxygen tension effects are valuable.

The nanofiber layers also could be used as a substrate for preservation and transportation of cells such as cryogenic preservation of tumors for transport. Aligned fibers can be used for nerve regeneration and promotion of axon growth; or for growing skin cells for grafting. The fibers could be used for any variety of personalized medicine where patient cells are removed from the body, plated onto the fibers, and specific therapies, treatments, or dosages are based on those patients' cells. The nanofibers could be made conductive to stimulate cell growth or differentiation.

Generation of three-dimensional tubes of aligned or unaligned fiber allows these desirable biological functions in a manner similar to that of hollow-wall bioreactor technologies. Combinations of this electrospun nanofiber with coatings derived from homogenized whole organs, organ-derived fluids or matrices surrounding specific cells associated with either (a) an organ of interest or (b) a disease of interest. The resulting three-dimensional matrix better recapitulates a specific organ or microenvironment of interest to the point of better targeting aspects of the in vivo microenvironment that correspond to individual patients.

Combinations of the electrospun nanofiber with coatings derived from a specific patient as a diagnostic tool to individualize treatment for said patient. The resulting three-dimensional matrix recapitulates a patient's own organ much more faithfully than any existing commercial product. Cells from the same patient can be cultured in this nanofiber matrix and monitored to assess characteristics of the cells in the diseased microenvironment as well as their response to specific therapeutic compounds.

Combinations of this electrospun nanofiber with coatings derived from homogenized whole organs, organ-derived fluids or matrices surrounding specific cells associated as an environment for organ- and patient-specific stem cell differentiation. The differentiation and conditioning of stem cells are controlled by these elements of the microenvironment to contribute to both the antigenic development and differentiation of pluripotent cells. On synthetic nanofiber matrices, these organ-based cues will provide a 3-D scaffold containing many of the biochemical and topographical compounds relevant to the ultimate goal of replacements generated from a patient's own cells.

These and many more uses and/or variations of the disclosed nanofiber layers will be appreciated by the skilled artisan based on the disclosure set forth herein.

### BRIEF DESCRIPTION OF THE DRAWINGS

For a fuller understanding of the nature and advantages of the present device, process, and methods, reference should be had to the following detailed description taken in connection with the accompanying drawings, in which:

FIG. 1 is a schematic of a completed multi-culture interactive microenvironment system (IMEMS) arrangement containing a representative population of different cell types relevant to cancer investigations. In particular, a Transwell® insert, 10, was placed in a well, 12, and filled with a culture media, 14. Insert 10 has a 0.4 μm pore size allowing for no cell penetration. A layer of large porosity fiber allowing for cell penetration, 16, was glued onto the bottom of insert 10. A second layer, 18, of small porosity fiber allowing no cell penetration was placed mid-way up insert 10. A layer of breast epithelial cells, 20, was placed in the bottom of well 12. Breast stromal cells, 22, were placed in contact with large porosity layer 16 and such cells penetrated layer 16. A layer of breast adipocyte cells, 24, was placed atop small porosity layer 18. Insert 10 had a hole, 26, drilled into it between the fiber layers for injection of cells into the insert.

FIG. 2 shows an exemplary electrospun polycaprolactone nanofiber electrospun onto conductive carbon tape strips (white, vertical bars). Note that, in this embodiment, the fiber bridges the bottom of each open well.

FIGS. 3A and 3B show the gross morphology (FIG. 3A) and MRI imaging (FIG. 3B) of a glioblastoma multiforme (GBM), the highest grade and most common primary glial tumor (52). FIG. 3A shows the aggressive, hemorrhagic tumor core as well as the extensive migration of glioma cells along aligned white matter (the corpus callosum) into the opposite hemisphere. FIG. 3B discloses the presence of both well-defined (high contrast, red arrows) and diffuse (poor contrast, yellow arrows) borders, the latter being the major regions allowing cell dispersion.

FIGS. 4A show glioma cells disperse along several anatomical structures such as myelinated tracts (1), blood vessels (91) and the basal lamina of the subpial surface (57). Periaxonal migration often results in perineuronal satellitosis (48).

Glioma cells can also move across the neural parenchyma and neuropil without apparent guidance from other structures. FIG. 4B Glioma cells detected on the abluminal surface of a solitary blood vessel within the tumor mass. FIG. 4C Glioma cells (arrows) detaching from the tumor core and invading the neighboring white mattercorpus callosum.

FIG. 5 is an artist's representation of the predominant molecules that compose the neural ECM close to the surface of neural cells. The CSPGs of the lectican family typically present a globular domain at each end and a stretched middle section decorated with chains of chondroitin sulfate. The illustration on the bottom left is an artist's impression of the mesh-like network of HA based on rotary-shadowed electron micrographs of this polysaccharide in aqueous solution. SGGLs: sulfo-glucuronyl glyco-lipids (a group of lipids enriched in the white matter that bind directly to CSPGs). Figure adapted from reference 75.

FIG. 6 is a flow Diagram showing the processing of human breast tissue as reported in Example 2 (below).

FIGS. 7A-7D depicts PHNBE's grown on electrospun fiber for 24 hours following treatment with FIG. 7A, 2.5% Z-sera; FIG. 7B, 50 nM Z; PHNBE's grown on CCPS for 24 hours following treatment with FIG. 7C. 2.5% Z-sera, and FIG. 7D 50 nM Z. CYP19 A1 Expression of Human Normal Breast Tissue on Electrospun PCL.

FIG. 8 graphically plots CYP19 A1 expression of human normal breast tissues following 4 days of culture on CCPS (‘PS’) controls, electrospun PCL (‘PCL’), and gelfoam.

FIGS. 9A and 9B are phase contrast microscopy observations of PHNBEs on (FIG. 9A) CCPS and (FIG. 9B) 40 micron-thick electrospun PCL (cells are indicated by arrows).

FIG. 10(A) is a SEM image of mammospheres seeded on electrospun fiber after 48 hrs. FIG. 10(B) is a SEM image of mammospheres seeded on CCPS after 48 hrs. FIG. 10(C) is the expression of cyclin D1, MMP3, Era and PTPRγ in mammospheres grown on electrospun fiber for 48 hrs.

FIGS. 11A and 11B are examples of isolated U251 cells seeded on aligned (FIG. 11A) or randomly oriented (FIG. 11B) nanofibers. Notice the remarkable elongation of the cell on the aligned nanofibers (center of the image). In contrast, cells on randomly oriented fibers did not shown any preferential extension.

FIGS. 12A and 12B are SEMs of as deposited, Random PCL nanofiber (FIG. 12A) and aligned PCL nanofiber (FIG. 12B). Scale bars: 10 μm.

FIGS. 13A and 13B are examples of isolated U251 cells seeded on aligned (FIG. 13A) or randomly oriented (FIG. 13B) nanofibers. Notice the remarkable elongation of the cell on the aligned nanofibers (center of the image). In contrast, cells on randomly oriented fibers did not shown any preferential extension.

FIGS. 14A and 14B are cytoplasmic GFP- and nuclear RFP-labeled U251 cells migrating on on random (FIG. 14A) and aligned (FIG. 14B) PCL fiber over a 24 hour culture period. Scale bars: 100 μm.

FIGS. 15A and 15B are cell motion tracking on as-deposited, random PCL nanofiber (FIG. 15A) or aligned PCL nanofiber (FIG. 15B).

FIG. 16A is a tracking of total cell motion (outset graph) and the average motion of 78 cells on random PCL nanofiber versus aligned PCL nanofiber over the 24 hour period. FIG. 16B is a tracking of two individual cells on aligned and random fiber. Cells on aligned fibers exhibit bursts of post mitotic motion (M, mitosis observed by the experimenter).

FIG. 17 is a photomicrograph of an example of an isolated glioma stem cell neurosphere seeded on randomly aligned fibers. Note the lack of cell detachment onto the supporting nanofiber.)

FIGS. 18A-18D are representative frames showing cell dispersion from glioma neurospheres seeded on aligned (FIG. 18A) versus random (FIG. 18B) electrospun nanofibers. The corresponding bounding ellipses (FIG. 18C and FIG. 18D) were estimated by principal component analysis. FIG. 18E is a graph showing the variation in the ratio of the elliptic axes (i.e., a direct measure of anisotropic cell spread) over time. Scale bars=100″m.

FIG. 19 shows PCL ‘core’ fibers covered by a ‘shell’ of hyaluronic acid (HA). Scale bar: 1″m.

FIG. 20 shows random PCL ‘core’+HA ‘shell’ nanofiber onto which a myelin+Dil solution has been printed in a horizontal pattern of stripes. Scale bar: 100″m.

FIGS. 21A and 21B are representative scanning electron microscopy images of mouse bone marrow cells plated on lung extract-coated nanofiber matrices. Cells began to adhere to the nanofibers after 2 days in culture (FIG. 21A). Mouse bone marrow cells (day 8) plated on a nanofiber matrix coated with PBS-treated lung extract coating. Cells are separated and do not appear to be secreting matrix materials (FIG. 21B). Mouse bone marrow cells plated on a nanofiber matrix coated with bleomycin-treated mouse lung extract. Cells tend to clump together and secrete more matrix materials on the bleomycintreated lung extract-coated nanofiber matrix.

FIG. 22 shows wild-type mouse bone marrow cells plated on nanofiber matrices coated with bleomycintreated lung extracts have increased expression of selected fibrotic genes after 8 days compared to cells plated on nanofibers coated with PBS treated lung extracts via quantitative real-time PCR. Expression of type I collagen, smooth muscle actin, connective tissue growth factor, and tenascin-C was significantly increased relative to GAPDH, a housekeeping gene (n=10 and *p<0.05).

FIGS. 23A-23C are scanning electron micrographs of wild-type BMSCs cultured on uncoated electrospun PCL (FIG. 23A), PCL (shell)/PES (core) (FIG. 23B), and PES fibers (FIG. 23C).

FIGS. 24A-24C are wild-type mouse bone marrow cells plated on the three different modulus nanofiber matrices coated with bleomycin-treated lung extracts. Increased expression of fibroblast/myofibroblast genes (type I collagen FIG. 24A, smooth muscle actin FIG. 24B, and connective tissue growth factor FIG. 24C) after 8 days was observed on the PCL/PES core-shell composition compared to cells plated on either PCL or PES nanofibers. Tenascin-C expression (data not shown) was statistically indistinguishable on the three compositions. (n=10 and *p<0.05).

The drawings will be described in further detail below.

### DETAILED DESCRIPTION

Using electrospun fiber layers, various embodiments provide an in vitro cell culture environment that may have topographical and spatial resemblance to physiological cell arrangements. Accordingly, exemplary embodiments may be much richer in terms of biologically important substances (e.g., cytokines, hormones, etc.) that can be produced by cells. An exemplary embodiment combines existing in vitro culture technology with at least two separate forms of electrospun fiber. For example, various embodiments may have one or more “high capacity” electrospun fiber layers (i.e., large porosity fiber that allows cell penetration). Additionally, various embodiments may have one or more “low capacity” standard electrospun fiber layers (i.e., small porosity fiber that does not allow cell penetration). The fiber layers may comprise aligned fiber layers (i.e., an electrospun fiber layer containing fibers generally having the same orientation), non-aligned fiber layers (i.e., a fiber layer having no standard fiber orientation), or a combination of both.

Other layers of varying porosity and thickness may be included as desired. The various electrospun fiber layers, of varying porosity, may be provided and arranged to achieve various topographical and physiological situations as desired. The spacing between the layers may vary from 0.0 to 10.0 cm depending on the interests of the investigators. Accordingly, various embodiments may be useful for cancer-based investigations of cell proliferation that potentially reflect on tumor occurrence and progression in vivo. Exemplary devices and systems may be extended to explore the influence of specific chemotherapeutics (either local or systemic) providing value as a screening tool for immediate clinical applications.

Although some embodiments are directed toward topographical and physiological resemblance, other embodiments are not so confined. Alternative embodiments may use electrospun fiber layers to achieve non-physiological or ultraphysiological cell culture arrangements that may be useful for various experimental or industrial purposes, (e.g., the layers may be arranged to achieve cell arrangements that yield ultra-heightened expression of a desired compound).

Exemplary embodiments may be used with almost any multi-well culture plate. Example embodiments may have electrospun fiber layers of varying porosity, shape, and thickness. These layers may be either permanently secured or detachably secured to the sidewalls of plate wells and/or well inserts. Furthermore, depending on the desired cell arrangements, many different fiber layer arrangements are possible.

An exemplary embodiment allows simultaneous culture of multiple cell types within or upon various electrospun matrices. In an exemplary embodiment, the arrangement of the electrospun fibers allows the cell populations to communicate chemically with each other. However, the submicron nature of the electrospun fiber layer may prevent physical contact of the cells.

Various embodiments may include the use of both “low capacity” standard electrospun fibers of at least one electrospun fiber layer having a high volume, three-dimensional porosity. With the addition of this “high capacity” layer, higher levels of proliferation of contact-inhibited cells and a corresponding increase in cell-cell ‘signaling’ may be accomplished.

In various embodiments, other non-fiber (e.g., plastic, gelatinous, etc.) layers may be utilized in conjunction with the fiber layers as desired for a given application. For example, the fiber layers may be imbedded within a gelatinous layer (e.g, Matrigel® BD Biosciences, San Jose, Calif.). In exemplary embodiments, non-fiber layers may be applied to the surface of the fiber layer, integrated with the fiber layer, or utilized as a separate layer in the microenvironment.

A preferred embodiment may be used in conjunction with Transwell® inserts or equivalent-type insert. Transwell® plate inserts (Corning Inc., Lowell, Mass.) have been successfully used to introduce more than one cell type into an existing well by separating the two populations with a track-etched membrane containing submicron pores. However, the advantages of electrospun fiber-topographical resemblance to the extracellular matrix surrounding human cells—may be lost. Well inserts, such as a Transwell® insert, provide a convenient platform for constructing apparatuses useful in various disclosed embodiments. Using these inserts, the cells may be conveniently loaded onto various and/or varying layers of electrospun fiber. In some embodiments, cell placement onto various layers may be accomplished by boring a small hole within the insert, a hole placed between individual fiber layers. Such a hole will allow specific injection of a cell type(s) onto a specific layer (electrospun fiber layer or otherwise).

However, it should be understood that embodiments do not require such an insert and various fiber layers (both high capacity and low capacity) may be secured directly into a well to achieve acceptable results. In such a case, cells may be loaded using other means, for example, one-way injection ports along the sidewall of the well.

Additionally, cell layer inserts may be designed as to stack on top of each other allowing a researcher to seed cells on one fiber layer, then add a fiber layer insert above the previous one and seed cells onto that layer. This process may continue for any number of stacked fiber layers.

Example embodiments may have immediate utility in oncology investigations such as cancer development and the influence of chemotherapeutic drugs on cancerous cells that can develop “chemoresistance” only in the presence of other cells. Furthermore, example embodiments may be useful in industry and science for increased or enhanced production of various biological products (e.g., cytokines, hormones, other desired biological products, etc.), because of the unique cell-cell interactions and growth relationships made possible by the various exemplary embodiments.

### Electrospinning Techniques

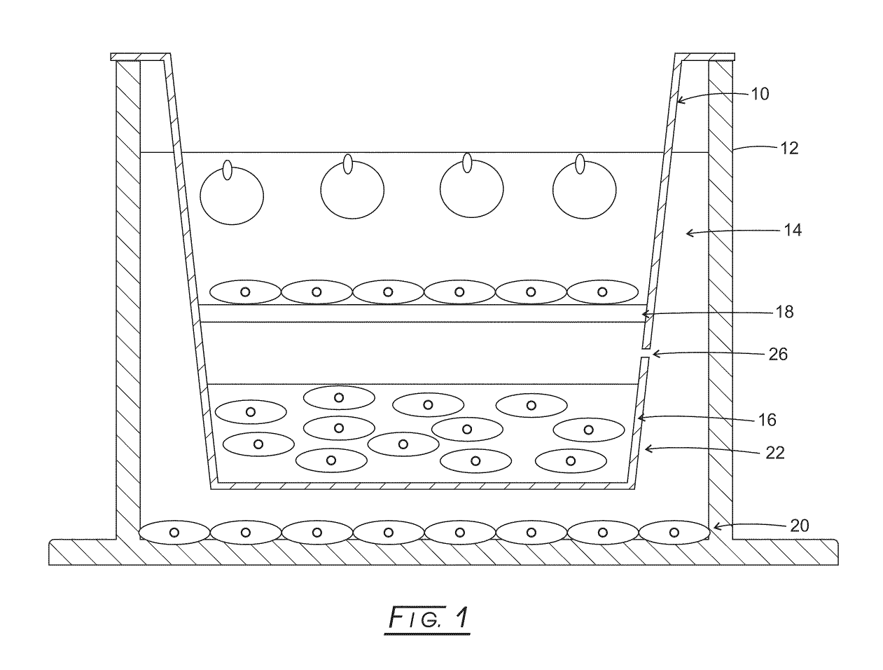

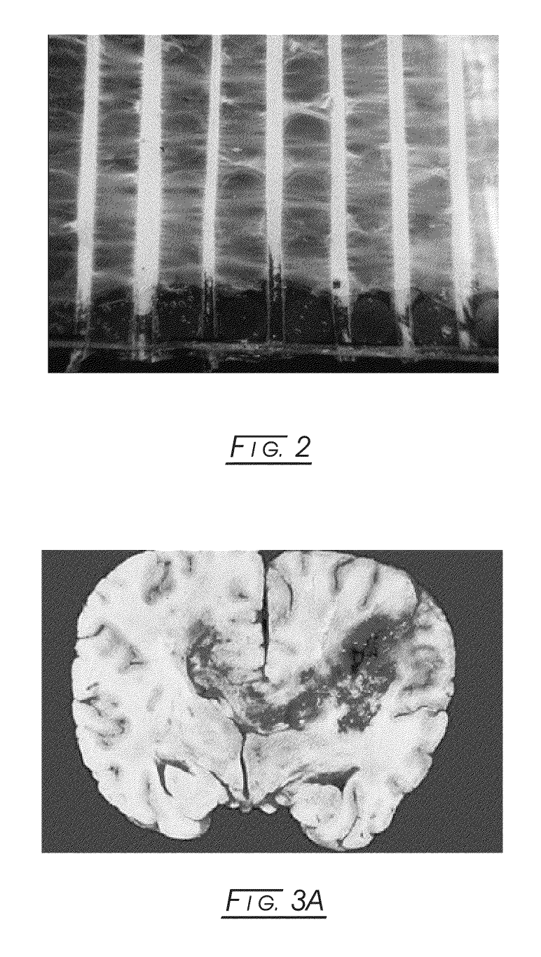

Electrospinning, a versatile technique producing either randomly oriented or aligned fibers with essentially any chemistry and diameters ranging from 15 nm to 10 μm (6, 7), has achieved broad applications in tissue engineering (8-22). Some prior work involving ‘soft,’ electrospun PCL has focused on the evaluation of mechanical properties (23-29), as modulus is known to be important in controlling the behavior of adherent mammalian cells. This level of familiarity with the literature has revealed that even for electrospun fiber the vast majority of such investigations are based on monocultures consisting of immortalized cell lines. The level of control over biological interactions afforded by a complex microenvironment call for scaffold designs that are more complex-and, thus, more biologically capable—than the standard practice of culturing “cells on a sheet.” The use of nanofiber in FIG. 1 confers a number of innate advantages over TCPS. Such nanofibrous environments have consistently shown more in vivo-like cell behavior (30-32) than standard 2-D or cell culture polystyrene surfaces and, thus, have the ability to conduct more biologically relevant studies of key biological processes.

If necessary, an even softer (lower modulus) polymer, such as polyethylene glycol, can be used to produce even less resistance to cell anchoring events. However, softer polymers typically (a) suffer from rapid water diffusion/infusion during cell culture and (b) exhibit substantial degradation. Both of these can create problems in regards to maintaining a constant modulus and chemical environment throughout a given experiment. Alternatives to polycaprolactone (PCL), a widely-utilized, FDA-approved biodegradable polymer that does not show significant changes in modulus during a several week exposure in vitro (39), should be pursued only if experimental evidence demands it.

### Manufacture of Nanofiber Layers

Exemplary embodiments include a very high volume process producing electrospun fiber substrates conveniently adapted to standard cell culture wells in multi-well plate formats (such as but not limited to 24, 96, or 384-well plates) at costs that are relatively small compared to the “cut and place” techniques described in the prior art. Various embodiments provide the biomedical community with an instantly recognizable, highly useful cell culture product having a very large market. Exemplary embodiments may be used in cancer-based investigations of migration that potentially reflect on invasion or metastasis in vivo. Furthermore, various embodiments may be used to explore the influence of specific chemotherapeutic drugs (either local or systemic) on patient-specific cancer cells providing value as a screening tool for subsequent clinical treatment of this patient.

One of the usual requirements of the electrospinning process is that the substrate upon which deposition takes place must be conductive in order to attract the falling fiber out of the air. In this context, aligned fiber that allows clear demonstrations of cell migration should be produced. Preferably a “split ground” deposition is used to achieve this arrangement, specifically adapted to this substrate. In an exemplary embodiment, this may be accomplished by adhering strips of conductive carbon tape in between the wells. Fiber then deposits alternatively between these strips producing aligned fiber at the bottom of the wells. Alternatively, this may be readily achieved with a manufactured carbon tape template adhered to the bottom of the empty wells.

Additional techniques for nanofiber formation include electrospinning a polymer-containing solution or a polymer melt onto a spinning/rotating drum/disk held at a different potential than the spray nozzle/tube to form aligned nanofibers, which then can be removed, cut to length and placed in a well or other container in single and/or multi layers, optionally with different sized nanofibers. Another technique is disclosed in U.S. Pat. No. 7,629,030. A variety of additional techniques are found in the literature and are used presently in a variety of industries. Such techniques can be applied in accordance with the precepts disclosed herein.

While a variety of research endeavors can benefit from the disclosed nanofiber layer environments, exemplary such endeavors will be discussed below as illustrative of such endeavors and not by way or limitation. The skilled artisan will appreciate a variety of additional such endeavors based on the instant disclosure.

### Oncologic Applications—Dietary Interaction

Tumors develop and progress in complex three-dimensional microenvironments in which both normal cancer cells encounter specific physical, chemical, and biological challenges. Following these exposures, some cancer cells display uncontrolled growth, invasiveness and the ability to metastasize. Others undergo apoptosis and disappear. These challenges constitute a critical event defining the eventual prognosis of patients who present with cancer. Because the interactions with between cells and the dietary components present in their microenvironments are dynamic, there has been intense interest in identifying mechanisms by which cells are influenced by these components. However, the role dietary influences play in the dynamic interplay between tumor cells and the physical and chemical challenges around them is uncertain. Part of the reason is that in vivo systems, and even complex tumor-derived matrices, do not allow for deconstruction of these parameters in a reproducible and rigorous manner.

The IMEMS nanofiber environment is believed to be quite useful in translating basic experimental data generated from laboratory experiments involving a solution to the problem of deconstruction. One of our targeted dietary components is zeranol (Z), a non-estrogenic agent with potent estrogenic action that is approved by the FDA as an anabolic growth promoter for use in the U.S. beef industry. The benefits of using Z in U.S. beef feedlots are improved feed efficiency, weight gain, and carcass quality. However, Z is considered a mycotoxin and an endocrine disruptor in humans. The FDA legal limit of Z is 150 parts per billion (ppb) in edible meat. One scientific, data-based hypothesis is that long-term low level exposure of biologically active Z metabolites (BAZMs) to women may pose an adverse health risk to estrogen sensitive organs such as the breast.

IMEMS is likely to offer revolutionary thinking to modify the conventional thinking of palatability and marbling in Z-containing beef products. It also is important to provide scientific-based information for FDA or USDA policy-making processes. According to the Congressional Research Service in 2000, more than 95% of U.S. beef cattle on large feedlots used growth promoters. The EU enacted a ban on importation of American beef products implanted with growth promoters in 1985. Despite rulings in 1997 by the WTO that EU's ban was illegal, the retaliatory policy of the U.S. Government, authorized by the WTO, imposed 100% tariffs on EU agricultural products. Even with the imposition by the United States, the EU still refuses to lift the ban. Recently, the USDA Food Safety and Inspection Service approved four non-hormone treated cattle programs in U.S. solely for export to EU markets. This program has caused domestic consumers to question why the government is not allowing them the opportunity to consume hormone free beef in the United States.

In the U.S., cottonseed oil (CSO) enters the human diet through its use in cooking, frying, and food processing. Another other dietary component of interest to study, (−)-GP, is present in salad dressing, shortening, margarine, canned or snack foods and chewing gum. Cottonseed meal (CSM) also has been used as a high quality protein supplement in the diets for meat producing farm animals, including aquatic fish. In 1974 the FDA limited the free (±)-GP content in cottonseed products to be no more than 450 ppm (0.045%) (33).

Significant evidence has shown (±)-GP's anti-proliferative effects against a variety of human cancer cell lines including those of the breast, ovary, cervix, uterus, adrenals, pancreas and colon (34-44). Reported data demonstrates the anti-proliferative effects of (±)-GP on MCF-7, MCF-7Adr and MDA-MB-231 human breast cancer cell lines (45, 46). Based on these published results, it is believed that the bio-active food components, (−)-GPCSO, may be capable of blocking the BAZMs-induced tumorigenic impact on human normal and cancerous breast cells, stem/progenitor cells isolated from human normal breast tissues, and stem/progenitor cells isolated from human breast cancer tissue (breast cancer stem cells).

The oil from conventional cottonseeds is a racemic mixture containing 65% (+)-GP enantiomer and 35% (−)-GP enantiomer. Selective breeding strategies have resulted in a novel cottonseed cultivar containing 65% (−)-GP enantiomer and 35% (+)-GP enantiomer. Previously published data (16) demonstrates that the (−)-GP has 10-fold the anti-human breast cancer proliferative potency than the racemic (±)-GP.

It has been demonstrated that (−)-GP is a potent natural small molecule demethylating agent capable of reactivating cancer suppressor genes in human breast cancer cells exposed to Z. Other related data entitled “Epigenetic Effect of Gossypol in the In vitro Suppression of Head and Neck Squamous Cell Carcinoma Cells was presented at the 2007 7^{th} International Conference on Head and Neck Cancer. These novel results are the first to demonstrate that (−)-GP is a much better demethylating agent than clinical therapeutic drugs currently being used (5-Aza-2′-deoxycystidine and Trichostatin A (TSA). These data demonstrate that (−)-GP can modify the DNA methylation of cancer suppressor genes in the CpG islands of the promoter regions to improve the therapeutic efficacy of human breast and head and neck cancer patients.

In this context, these two compounds represent (a) a potential dietary addition ((−)-GP) and (b) a potential dietary deletion that could reduce the incidence of human breast cancer. By combining tools that physical scientists can bring to bear with a source of primary cells from cancer patients already predisposed to tumor development, we can propose a unique system—the IMEMS—allowing studies of both diet and cell-cell communication on specific cancer-related processes (proliferation, angiognesis, invasion, apoptosis, and metastasis). The emphasis on dietary influences must be accompanied by systematic variation of the surrounding microenvironment to better understand how cells interact with each other and with the physical and chemical nature of their surroundings. Tools developed pursuant hereto have particular relevance as they can (1) present relevant nanoscale features to seeded cells, (2) incorporate specific physical and chemical elements of the tumor microenvironment, (3) incorporate cell-cell communication to establish whether seeded normal or tumor cells undergo transitions that ultimately contribute to enhanced tumor severity or metastasis. Such tools can be used to examine the interplay between the physical and chemical parameters of the microenvironment and cell-cell communication as reflected in the growth of highly relevant human primary cells.

### Brain Oncology

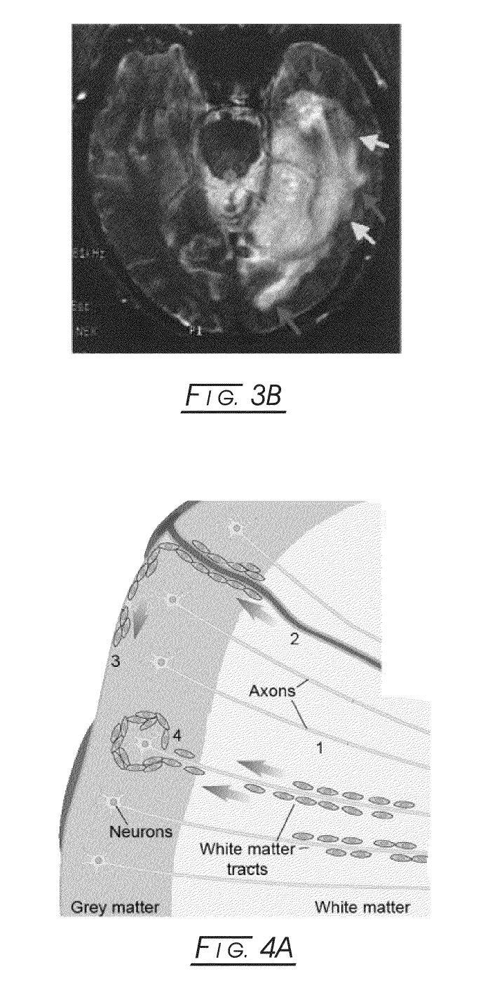

Malignant gliomas are among the most aggressive and least successfully treated types of cancer; few patients survive longer than a year following diagnosis (48, 49). This bleak prognosis is due in large part to the uniquely invasive ability of glioma cells, which allows them to detach from the tumor mass, infiltrate normal brain tissue, evade immunodetection and resist normally cytotoxic therapies (50, 51) (FIGS. 3A and 3B). Dispersion prevents complete surgical removal and contributes to recurrence and a rapid, lethal outcome.

While most therapeutic strategies focus on killing proliferating cells in the main tumor mass or starving the tumor core using anti-angiogenic approaches, few attempts have been made to target the non-dividing, migratory cells that cause tumor infiltration (50, 52). Approaches targeting these cells are greatly hampered by the difficulty in modeling glioma cell migration appropriately in vitro. While some labs have considerable expertise in 2D and 3D assays traditionally used to analyze glioma migration, they have found significant limitations restricting their potential to predict behavior in vivo [see “Modeling Glioma Cell Migration in vitro, below]. A major research goal, then, is to establish a nanoscaled in vitro model allowing a large degree of experimental control while accurately recapitulating glioma cell behavior(s). One proposed model is a well-defined in vitro assay that meets these specific needs. The model also allows cell accessibility for subsequent biochemical, genetic and microscopic imaging analysis. These levels of control and accessibility are challenging to impossible in other migration models (described below).

By the time of diagnosis and treatment, these glioma cells have invaded the surrounding brain tissue to an extent beyond what can be surgically resected (53-56), meaning that the only hope for a cure is likely a nonsurgical one. According to the recently issued NIH's Program Announcement PAS-08-048 (“Understanding and Preventing Brain Tumor Dispersal”), novel strategies against glioma invasion should be aimed at 1) identifying the mechanisms that mobilize tumor cells, 2) determining how motility is affected by interaction of the tumor cells with normal brain elements and 3) translating understanding of those parameters to viable interventions against invading cells. Accordingly, we believe that this proposal will generate nanoscale strategies having direct relevance to the development of reagents tailored to target invasive mechanisms of gliomas and transform these tumors from a death sentence into a manageable disease.

Glioma Migration In The Brain

The invasive ability of gliomas is restricted to the CNS and involves both short-range, non-oriented invasion through the neural extracellular matrix (ECM) as well as long-range invasion along the major axis of elongated structures such as blood vessels and white matter fibers acting as “highways” for glioma dispersion (57, 58) (FIGS. 4A, 4B, and 4C). The invasive behavior of gliomas can be detected in both low- and high-grade tumors, indicating that invasive ability is acquired early in tumorigenesis. This may be an inherent property of these cells reflecting the cell of origin, likely a neural or glial progenitor cell or an immature astrocyte that has undergone de-differentiation. These patterns of invasion are highly variable between patients and likely depend on specific genetic changes in each tumor, the tumor stem cell of origin and tumor localization. Understanding how these factors affect the invasion process is a major reason for developing more representative in vitro models.

Stereotyped patterns of invasion and the fact that they only invade neural tissue (59, 60) suggest that a combination of glioma-specific molecular mechanisms and the unique composition of the neural microenvironment underlie dispersion in the CNS. Accordingly, a representative model of glioma invasion should pay close attention to both substrate topography and chemical signals in the neural microenvironment that may influence migration of these cells. In particular, this model should reproduce some of the major features observed in real gliomas such as preferential invasion along the major axis of anatomical structures, the presence of white/grey matter boundaries, and haptotactic gradients of extracellular molecules that can influence glioma cell motility and proliferation.

Modeling Glioma Cell Migration in Vitro

Some of the most common models used to study glioma cell motility rely on bi-dimensional assays, such as the “wound-healing” assay in dense cultures and transwell motility tests to study chemotactic or haptotactic effects. While informative, these assays have serious drawbacks that stem from the use of cells arranged in flat monolayers on top of a bulk (usually plastic) substrate that is both homogeneous in topography and highly rigid. This condition induces glioma cells to adopt fibroblast-like morphologies quite different from those found in 3D assays as well as in vivo (61-63). Both cell migration and proliferation are affected by this substrate in a manner that is difficult to interpret and compare to the behavior of real gliomas.

Another common set of assays used to study glioma cell invasion is based in the penetration of glioma cells through a matrix usually composed of type I collagen or combinations of this fibrillar protein and additional fibrillar proteins such as laminin and nidogen (e.g., the Matrigel matrix). These assays have proved useful in studying certain aspects of glioma invasion, such as, the effects of proteases in cell dispersion (64, 65), but suffer from limitations in design. For instance, the homogeneity of these substrates results in non-directional migration influenced only by external chemoattractants (if used). In addition, the major component of these matrices, fibrillar collagen, is not only absent from the neural parenchyma and white matter fibers but is only present in scant amounts in the basal lamina of brain blood vessels. This, combined with the fact that glioma cells do not degrade the vascular basal lamina and do not intravasate in the brain, limit the relevance of these models in interpreting the infiltrative behavior of real gliomas.

Finally, one of the most realistic in vitro models of glioma cell dispersion (i.e., the combined effects of motility and substrate degradation) is the analysis of glioma cells placed on brain slices supported with appropriate culture media. (66-68). In these assays glioma cells are challenged to migrate through living neural tissue that retains most of the brain cytoarchitecture, including its natural barriers to cell movement (69). These assays, however, are cumbersome, extremely time consuming and present additional limitations in monitoring the cells as they move in a gradually dying substrate that may affect the glioma cells as the experiment proceeds.

The aligned fiber multiwall plate is believed to overcome these limitations and provides a number of significant advantages having clear clinical potential. First, from a technical standpoint, the electrospun fiber model retains all the advantages of a defined in vitro system coupled to the convenient monitoring of glioma cells in 3D using time-lapse confocal microscopy. Second, the current nanofiber composition—polycaprolactone (PCL)—forms a self-sustaining 3D scaffold and does not require the addition of gelling molecules that may stimulate artificial cell behaviors. Third, modifications allow the inclusion of relevant molecules found in the extracellular space of the neural tissue, potentially forming “shells” around the nanofibers that mimic the composition of blood vessels, myelinated tracts and random ECM fibers. Moreover, this model allows researchers to combine these biocoated electrospun fibers with additional printing of molecules on top of the original fibers; thus, permitting the study of haptotactic gradients having well-defined boundaries. Finally, these gradients can be used in combinations of random and aligned fibers, allowing a higher degree of control of topographic and chemical signals than in any previous model. From a design standpoint, the electrospun fiber model allows complexity surpassed only by the architecture of the brain itself while providing a level of control found in simple 2D assays.

Brain Microenvironment I: The Neural Extracellular Matrix

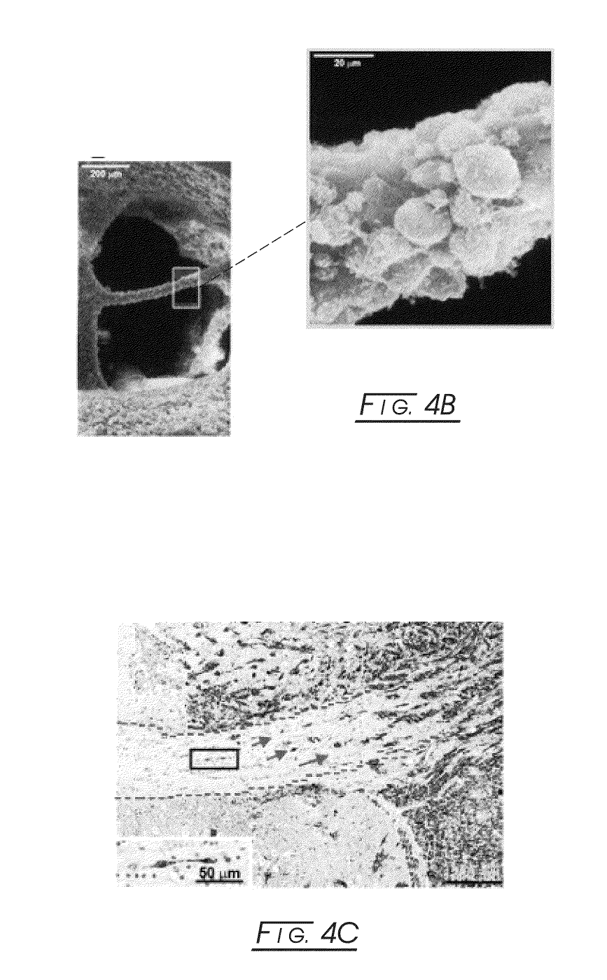

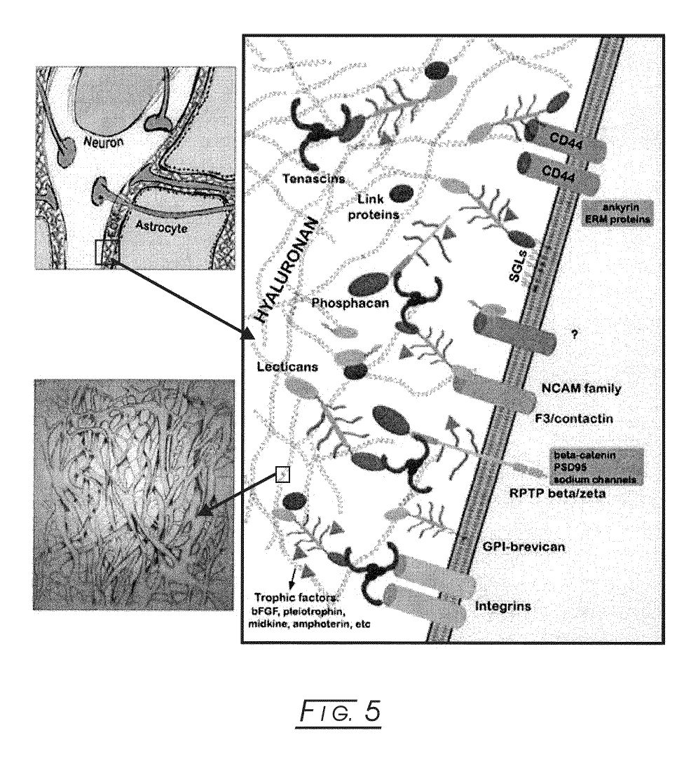

The neural ECM comprises as much as 20% of the adult brain volume and envelopes all structures within the neural parenchyma (70). This matrix is composed of a scaffold formed by the polysaccharide hyaluronic acid (HA) and associated glycoproteins and proteoglycans (FIG. 5), but is devoid of fibrillar proteins (collagens, laminins) supporting cell motility (70, 71). Indeed, the neural ECM forms an inhibitory terrain for cell migration, with a major “barrier effect” attributed to the abundance of glycoproteins bearing chains of the negatively charged polysaccharide chondroitin sulfate (chondroitin sulfate proteoglycans or CSPGs) (72-74).

While the composition of the neural ECM is complex and involves many types of glycoproteins (3, 33), both HA and the CSPGs are the key components that determine the overall structure and physicochemical properties of this matrix (75-77). HA is an extremely large (>106 Da/molecule) glycosaminoglycan (GAG) that can retain large amounts of water and, thus, creates hydrated spaces used by cells to proliferate and migrate. This GAG is mostly water-soluble in the developing CNS but (in the adult brain) becomes associated with proteoglycans forming large, water insoluble aggregates that reduce the interstitial space and gradually change the neural ECM from a permissive to a restrictive environment for axonal navigation or cell motility (77-79).

The CSPGs of the lectican family are the major group of aggregating proteoglycans that bind and organize HA in the adult ECM (77). These large, heavily glycosylated proteins carry chains of chondroitin sulfate, another GAG that is negatively charged at physiological pH and can act as an ionic buffer and as a molecular trap for small soluble trophic factors (80-83). Several lines of evidence have shown that the CSPGs are highly inhibitory to cell migration in vivo and in vitro, an effect that seems to depend mostly on chondroitin sulfate chains rather than on the core proteins (72, 74, 84). Together, HA and its associated CSPGs form a highly compressible mesh that embeds the glioma cells in the grey matter and restrict (or should restrict) their motility. However, glioma cells efficiently overcome this barrier to cell motility, thanks to their ability to degrade the normal matrix (85, 86) and secrete their own matrix components (87, 88, 89).

Brain Microenvironment II: Basal Lamina and Myelinated Tracts

As indicated above, the neural ECM forms a randomly organized mesh of small, highly compliant fibers 0.5 to 3 μm in diameter that do not provide anchorage points for the formation of intracellular stress fibers in motile cells. Thus, the major routes of dispersion of gliomas do not involve traversing grey matter but rather migration over larger structures that provide both the physical resistance and molecular anchors that permit the formation of focal adhesions and thus facilitate cell motility. The two major structures co-opted by glioma cells for this purpose are the brain capillary network (ranging from 5 to 10 μm in diameter) and the major bundles of myelinated axons (approximately 0.5 to 3 μm in diameter (90)) that constitute white matter. Dispersion along these pathways gives rise to characteristic “chains” of elongated glioma cells around blood vessels and nerve fibers, known as pen-vascular and peri-axonal satellitosis, respectively (51, 56). A late pathway of dispersion using the subpial space is likely a variation of the hematogenous dispersion and is observed when glioma cells moving along blood vessels finally reach the internal surface of the pia mater.

Both the composition and shape of the brain blood vessels are known to influence glioma cell motility and proliferation. In particular, the presence of gaps and branches disrupting the major axis of migration slow glioma cells and stimulate division, illustrating the importance of substrate topography in controlling glioma migration (91).

The composition of the brain blood vessel ECM, or basal lamina, is quite distinct from the neural ECM and comprises several fibrillar proteins known to strongly promote motility, such as the laminins and, particularly, the large pro-adhesive and pro-motility glycoprotein fibronectin (FN). Interstitial collagens such as type I collagen are for the most part absent but this ECM is rich in non-fibrillar collagens such as type IV. Of the different components of the basal lamina, FN has been identified by several lines of evidence as one of the major haptoattractants for integrin-dependent glioma cell migration (92, 87, 93).

While the composition of the neural ECM and the basal lamina are relatively well known and the motile behavior of glioma cells on these microenvironments has been previously analyzed, the major pathway of glioma cell dispersion, i.e., the white matter, remains poorly studied. Gliomas disperse along white matter fibers more than on any other structure in vivo, even reaching the opposing brain hemisphere over the course of a few months (103). The directional migration along white matter fibers has traditionally been regarded as an anatomical “pathway of least resistance,” where glioma cells follow the axis of myelinated axons but the molecular mechanisms that allow glioma dispersion on this substrate are for the most part unknown (104, 105). Moreover, the white matter from the CNS is known to be a non-permissive environment for process extension (106, 107) and some of the most conspicuous molecular inhibitors, such as myelin associated glycoprotein and the NOGO protein, can inhibit neural cell migration (108, 109, 110). Indeed, previous in vitro studies have reported a lesser migratory capacity of cultured glioma cells on white matter components than on basal lamina components (105-106, 111), in contrast to the observed behavior of gliomas in vivo.

In large part, understanding the influence of white matter on glioma migration has been hampered by the laborious process of CNS myelin extraction and the difficulty of dealing with a complex mixture that is ˜70% w/v lipids. However, another important deficiency of previous studies has been the unavailability of a directionally oriented substrate on which to deposit purified myelin. Myelin-coated plastic wells challenge glioma cells with a homogeneous substrate eliminating the non-random component of glioma migration on white matter that predominates in vivo. By preparing a highly purified myelin suspension that can be co-spun with the PCL during electrospinning of nanofibers, we have overcome these limitations and produced an aligned distribution of myelin molecules on a nanofiber substrate, mimicking the natural topography.

### Fibrosis in Asthma

Asthma is a syndrome initially characterized by reversible lung obstruction, hypersensitive airways and airway inflammation. However, as the severity increases, airway fibrosis ensues and the airway obstruction becomes irreversible. Approximately 34 million patients are diagnosed with asthma, with an associated 250,000 deaths/year. Asthma accounts for ˜500,000 hospitalizations/year, 12.8 million missed school days/year and 10 million missed work days/year. The annual cost of asthma care is $19.7 billion/year and the associated annual drug costs are over $6 billion/year. Despite improvements in our understanding of the fundamental mechanisms of asthma, a complete understanding of the role of specific cells in the disease progression, genetic susceptibility, and the signals that induce airway fibrosis remains elusive.

Asthmatic Airway Fibrosis

In patients with asthma inhaled allergens and particulates find their way to the inner airways and are ingested by antigen-presenting cells (APCs). APCs then “present” pieces of the allergen to other immune system cells. The resultant TH2 cells activate the humoral immune system. The humoral immune system produces antibodies against the inhaled allergen. Later, when a patient inhales the same allergen, these antibodies recognize it and activate a humoral response. Inflammation results as compounds are produced that cause the wall of the airway to thicken, cells which produce scarring to proliferate and contribute to further airway remodeling. Mucus-producing cells grow larger and produce more and thicker mucus, and the cellmediated arm of the immune system is activated. Inflamed airways are more hyper-reactive and more prone to bronchospasm. Ultimately, the cells recruited to the airway produce collagen, leading to fibrosis. Therefore, it is imperative that the cellular and molecular mechanisms of asthma pathogenesis are elucidated so that new targets for potential therapeutics may be discovered.

The Role of Myofibroblasts in Airway Fibrosis

Excessive collagen deposition, myofibroblast expansion, and the development of fibrotic foci are hallmark pathological events in asthma. The myofibroblast is the key effector cell in this process, as it is responsible for the synthesis and deposition of collagen and other inappropriate matrix materials that lead to overabundant smooth muscle mass associatied with airflow obstruction (112). The origin and mechanism of recruitment of the myofibroblast is unknown, however there is exciting recent advances in the mechanisms underlying fibrosis. This new research shows that myofibroblasts may be derived from bone marrow-derived circulating cells known as fibrocytes (113-115). Discovered in 1994, these cells, which express type-1 collagen, are derived from the bone marrow (116). Recent studies have demonstrated that fibrocytes traffic to the airway during asthmatic injury (112, 117, 118). However, the mechanism by which fibrocytes differentiate into fibroblasts and myofibroblasts and the lineage of these cells is poorly understood. The goal of this proposal is to generate an ex vivo system that will allow us to determine the source of the myofibroblasts in airway inflammation. This is important because while there is evidence that each of the proposed mechanisms are possible, it is necessary to determine the origin of myofibroblast precursors and the factors that mediate their recruitment, as this could lead to promising new therapeutic targets.

The Fibrotic Microenvironment

While it is important to study the individual cells responsible for mediating subepithelial lung fibrosis under ‘normal’ in vitro conditions, in vivo these cells exist in a far more complex microenvironment. Therefore, it is imperative to understand the role of the airway microenvironment in promoting fibrotic processes. The microenvironment is an intricate network of both structural and inflammatory cells, cytokines, proteins, and growth factors. The airway consists of resident structural cells such as epithelial cells, fibroblasts, and resident smooth muscle progenitors and resident phagocytes such as airway macrophages (119, 120) and neutrophils. The interactions between these cells and fibrotic factors during the pathogenesis of airway fibrosis are poorly understood. As discussed above, fibroblasts and myofibroblasts play an important role in creating a fibrotic environment, as they secrete excess collagen and matrix materials that lead to irreversible scarring. Cell-tocell adhesion molecules and extracellular matrix ligands are important factors in the fibrotic microenvironment, and several studies have investigated their role in promoting fibrosis and fibroblast differentiation (121-126). Adhesion-mediated signaling has become an area of intense study, as recent data indicates that cell differentiation and migration occurs in response to mechanic cues from the microenvironment, such as stiffness of the surrounding matrix (127-132). In a recent study published in Cell by Engler and co-workers, mesenchymal stem cells (MSCs) were cultured in matrices of different elasticity (modulus). Soft matrices resulted in differentiation of MSCs into neuron-like cells, whereas stiff matrices were myogenic (133). These studies underscore the importance of the extracellular matrix in directing cell fate and migration. With our novel ex vivo system, we will be able to dissect out various factors that contribute to myofibroblast differentiation, which would be incredibly difficult to accomplish using standard in vivo models.

Use of IMEMS in Asthma Fibrosis Research

A synthetic nanofiber matrix partially generated from murine asthmatic airway homogenates capable of inducing the differentiation of murine bone marrow-derived stem cells has been produced. To test the hypothesis that the airway microenvironment changes dynamically during the initiation, progression and resolution of asthma while causing the differentiation of bone marrow-derived stem cells (BMSCs), lungs from murine ovalbumin models will be homogenized at timepoints correlating to the initiation, progression of disease and end-organ fibrosis resulting from asthma and deposit the homogenate onto a coated nanofiber matrix. BM cells will be plated on the matrix and assessed for fibrotic gene expression and (myo)fibroblast differentiation, including collagen I, smooth muscle actin (SMA), tenascin-C (TN-C) and connective tissue growth factor (CTGF) expression by real-time PCR. Total collagen will be assessed by Sircol assay, and protein expression analyzed by immunohistochemistry and Western blots. This hybrid biologic-synthetic in vitro assay can potentially work with patient-derived tissues to overcome typical limitations and better determine patient outcome.

To establish the effects of this model asthma microenvironment on relevant human derived cells, assess human immune and airway cells (from the blood, lung via bronchoalveolar lavage (BAL), and the respiratory tract) will be assessed. This analysis also will focus on co-culturing the immune cells with primary cells from lung epithelial, endothelial, and fibroblast sources to understand the impact of the coated nanomatrix on gene and protein expression. The resulting in vitro models are a more effective means of understanding disease progression than information gathered from either single cell suspensions or respiratory tract tissue explants. Research, then, also will interrogate lung samples from patients with specific asthma types (mild, moderate and severe persistent asthma) to identify the gene and proteins uncovered in the in vitro studies to in vivo lung samples.

The following examples show how the disclosed developments have been practiced. Such examples are not a limitation on the disclosure, however, but rather are an illustration thereof.

### Example 1

A 5 wt-% solution of polycaprolactone (PCL) in 1,1,1,3,3,3-hexafluoro-2-propanol (HFIP) solvent was prepared by continuous stirring at room temperature to dissolve the PCL. The solution then was placed in a 60cc syringe with a 20-gauge blunt tip needle and electrospun using two high voltage DC power sources. One power source was set to −11 kV and connected to a rotating wheel, the other power source was set to +14 kV and connected to the needle. A copper loop then was attached to the needle to focus the fiber towards the wheel. The distance between the needle tip and the wheel was set to 20 cm. Using a digital tachometer to measure the wheel revolutions per minute and knowing the outer diameter, the wheel surface velocity was set to approximately 15 m/s to create aligned fiber or approximately 0 m/s to create random fiber. A syringe pump was used to supply the solution from the syringe at 15 mL/hr.

Fiber was deposited directly onto the metal surface of the wheel or to a thin polymer film that has been wrapped around the wheel surface until the desired thickness was achieved. The polymer film with deposited fiber or the fiber mat then was removed from the wheel and cut into the appropriate dimensions. These pieces then were glued or bonded to the bottom of a multiwell plate. After allowing the glue to cure, the plates were sterilized by soaking in 70% ethanol for 30 minutes to 12 hours before cell culture.

### Example 2

### Multi-Culture Embodiment Utilizing a Transwell® Insert

A commercially available Transwell® insert, like that shown in FIG. 1 or equivalent plate well inserts, may be utilized as a platform for constructing apparatuses useful in various embodiments.

One of the inherent limitations of standard electrospinning is that it typically produces cell impermeable membranes. Various embodiments overcome this limitation by spinning a high solids content fiber shown to allow full penetration by seeded cells (see references below), providing much higher cell ‘capacity’ than standard, ‘two-dimensional’ electrospun fiber. In this example embodiment, fifteen percent poly(caprolactone) (PCL, MW 65,000; Sigma-Aldrich, St. Louis, Mo.) dissolved in dichloromethane (Mallinckroff Baker, Phillipsburg, N.J.) was electrospun onto an aluminum foil—wrapped 7.6 cm×7.6 cm steel plate at −20 kV with a flow rate of 15 mL/h and a 30 cm tip-to-substrate distance. A voltage of 0 to +5 kV was gradually applied to the ground plate over the course of 1.5 h of electrospinning to compensate for the effect of insulation caused by the gradual thickening of the deposit. The as-spun fiber mesh was treated in a vacuum oven (<30 mmHg) at 45° C. for 24 hours to remove residual solvents. The approximately 3 mm-thick mesh then was cut into 6 mm-diameter cylinders using a biopsy punch (Miltex, York, Pa.).

Appropriately sized cutouts which correspond to the Transwell® insert (in this example, circles) of this “high capacity” fiber layer may then be added to the insert above the bottom membrane and then secured (e.g., glued) into place as shown in FIG. 1.

Standard electrospinning then may be engaged to form a continuous sheet of standard electrospun fiber. From this sheet, circles of fiber of a diameter that fits the bottom of the Transwell insert are cut and these cut-outs are then secured into place above the previously inserted “high capacity” layer. In FIG. 1, an embodiment comprising a “low capacity” standard electrospun fiber is glued into place above the previously inserted “high capacity” layer shown in FIG. 1.

A small hole may be drilled into side of the insert between the two layers to allow for seeding of cell populations into the lower, “high capacity” layer. In FIG. 1, a needle may be inserted into the intervening space between the two fiber layers of a completed insert to seed cells into the high capacity fiber layer.

Once the insert is completed, all that remains is to add the insert to an existing insert adapted plate to achieve the biological interactions shown, as for example, in FIG. 1. FIG. 1 is a schematic example of a completed interactive multi-culture microenvironment system arrangement containing representative populations of different cell types relevant to cancer investigations. In this example embodiment, three cell layers are found: (1) the bottom of the plate well (which can be either standard tissue culture polystyrene, electrospun fiber, or any other well material known in the art); (2) the “high capacity” fiber layer; (3) the standard, “low capacity” fiber layer. In addition, in this context fat globules (floating pink spheres) were added to further simulate critical aspects of human biology in cancer development.

### Tissue Procurement and Processing for Cell Seeding

Human breast reduction from normal (˜220 gram) and cancerous tissues (˜0.5-1 gram) were obtained through the Tissue Procurement Program at The Ohio State University Comprehensive Cancer Center in Columbus, Ohio. Tissues were dissociated overnight on a rotary shaker at 37° C. After dissociation, the dissociated tissue was centrifuged for 5 min, 700 rpm in 50 ml centrifuged tubes and the resulting pellets, which were highly enriched with epithelial organoids, were washed several times with PBS with 2% Fetal Bovine Serum (FBS) and centrifuged at 1,200 rpm in 50 ml centrifuged tubes after each washing. 1-5 ml of pre-warmed trypsin-EDTA was added to the organoids pellet and was pipette with P1000 for 3 minutes, and then 10 ml of cold PBS with 2% FBS was added and centrifuged at 1,200 rpm for 5 min. After centrifugation, supernatant was removed, and 2-4 ml of pre-warmed dispase and 200-400 μl of 1 mg/ml DNAse 1 was added and pipetted for 1 min. 10 ml of cold PBS with 2% FBS was added and the cell suspension was filtered through a 40-μm cell strainer to obtain a single cell suspension. Cells were cultured in suspended in MEBM supplemented with 1× B27, 20 ng/ml EGF, 1 ng/ml

Hydrocortisone, 20 mg/ml Getamycin, 5 mg/ml Insulin, 100 mM 2-mercaptoethanol and 1% antibiotic-antimycotic (100 units/ml penicillin G sodium, 100 mg/ml streptomycin sulfate and 0.25 mg/ml amphotericin B) in a 37° C. humidified incubator (5% CO₂: 95% air). FIG. 6 illustrates the detailed protocols routinely employed in the laboratory for the isolation of human breast epithelial cells, stromal cells, pre-adipocytes, and lipid droplets from human normal and cancerous breast tissues. We can use these cells in the proposed studies as either a single cell type monolayer culture on 2-D polystyrene plastic surface or added to a 3-D nanofiber PCL matrix. Isolation methods providing different breast cell types from human normal and cancerous breast tissues have been published by our laboratory (47, 134-136). The experimental data generated from different human normal and cancerous breast cells and stem/progenitor cells and breast cancer stem cells cultured on the 2-D polystyrene plastic monolayer and will be compared to the results produced from the single cell type cultured on bio-mimetic 3-D nanofiber PCL and multiple cell types culture in the newly designed IMEMS (FIG. 1) model system.

In FIG. 6, tissue, 100, is minced, and placed in a dish, 102, followed by collagenase digestion, 104. Tissue 100 next is centrifuged, 106, producing layers (from top bottom) of lipids, pre-adipocytes, stromal cells, and organoids/epithelial cells. Separately, the lipids and pre-adipocytes are sent to culture, 108, in high Ca²⁺ (1.05 mM) and DMEM/F12. The stromal cells and organoids/epithelial cells are washed 5 times, and allowed to settle by gravity, 110. Stromal cells are sent to culture, 112, in high Ca²⁺ (1.05 mM) and DMEM/F12. The organoids/epithelial cells are sent to culture, 114, in low Ca²⁺ (1.05 mM) and DMEM/F12.

To our knowledge, this tissue capability allowed us to make the first attempt to quantify the net effect of interactions between different primary human breast cells using a multi-cellular system to generate cellular and physiological changes in an experimentally controlled microenvironment. We fully anticipate that certain, as yet undefined factors (either stimulatory or inhibitory) may be secreted into the culture medium from one or more of the four types of cells. The conditioned medium harvested from the IMEMS could serve as a source of potential biologically active factors useful for discovering novel therapeutic agents to treat or prevent human breast cancer.

### Primary Cultured Human Normal Breast Epithelial (PHNBE) Cells on Electrospun Fiber

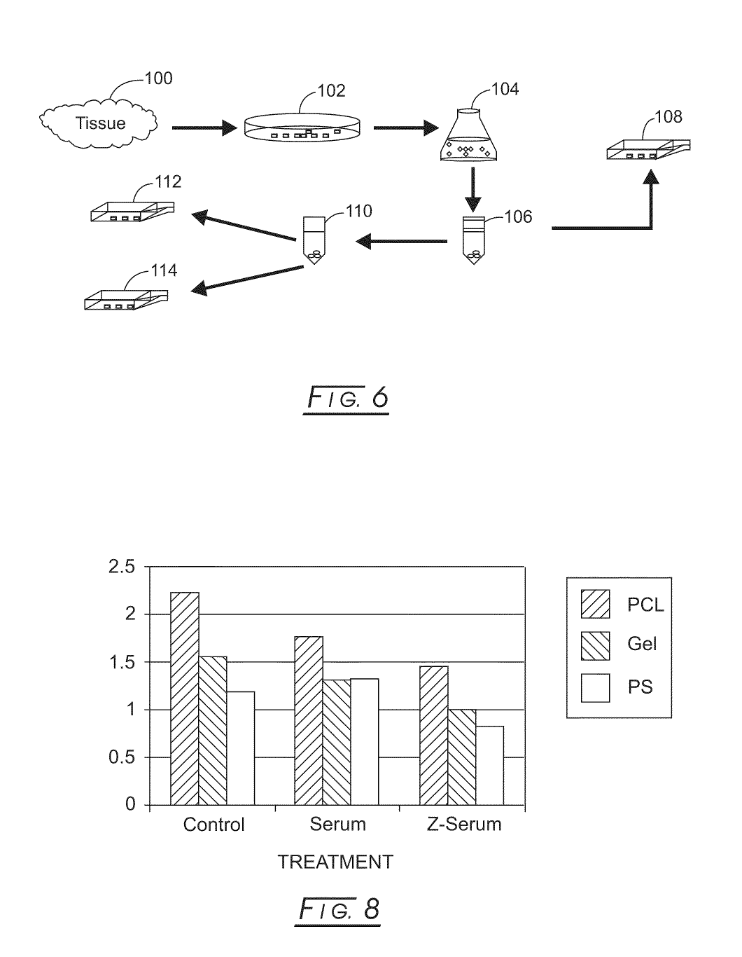

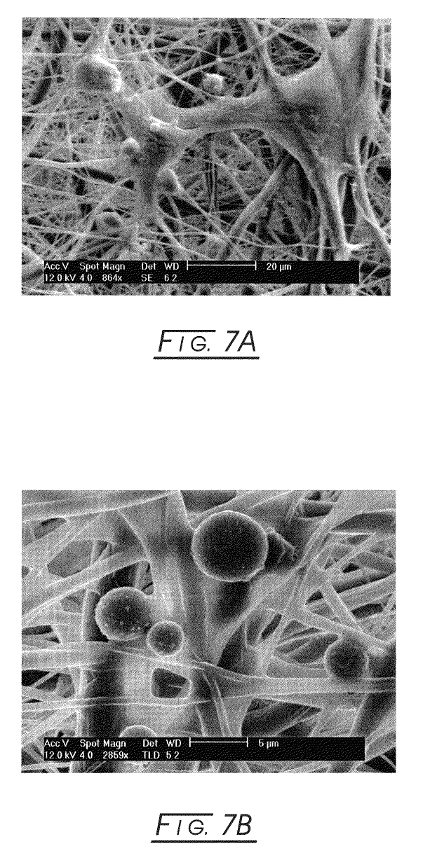

Our preliminary study illustrates the interesting and distinct biological response of control and treated PHNBE cultured on a single layer electrospun PCL, the material making up the novel IMEMS. Scanning electron microscope (SEM) images in FIGS. 7A-7D show PHNBEs cultured on 3-D PCL nanofibers exposed to 50 nM of Z and 2.5% Z-sera for 24 hrs. The upper panel micrographs are low magnifications of 3-D cell morphology for each treatment group. The lower panel micrographs are taken from TCPS. The morphology of PHNBEs cultured on 2.5% Z-sera is visibly different from that on the TCPS. The prominent spreading of cells with extended processes observed in the PHNBEs treated with 2.5% Z-sera indicates a transition of the cells to an epithelioid morphology. It is likely that the cellular and molecular functionalities of PHNBEs grown on 3-D PCL electrospun fibers more accurately represents in vivo human body physiological conditions compared to the same cells grown on conventional plastic polystyrene matrix.

In an additional test of the differences between electrospun fiber and TCPS, the expression of the aromatase gene, CYP19 A1, was established in human normal breast tissues cultured in sera for 4 days on 3-D PCL nano-fibers, TCPS controls and Gelfoam (Pharmacia & Upjohn).

As shown in FIG. 8, exposure to 2.5% control serum and 2.5% Z-serum resulted in higher CYP19 A1 mRNA expression compared to the same exposure of breast tissues cultured on TCPS. CYP19 regulates aromatase enzyme production; aromatase plays an important role in the biosynthesis of estrogen that is thought to play a key role in breast cancer development. The functionality expressedin CYP19 A1 by human breast tissue culture on bio-mimetic 3-D PCL nano-fibers better simulates that shown in normal physiological conditions in vivo than on the other 2 matrixes. Exposure of human breast tissues in organ culture for 4-day on the 3-D-PCL matrix also resulted in distinctly different to impact the functionality of aromatase gene expression level. This data further supports our hypothesis that 3-D PCL matrix can exert a more biological influence not only on cell-to-cell level but also on the organ culture levels versus TCPS.

### Direct Observation of Primary Cultured Human Normal Breast Epithelial Cells (PHNBEs) on Electrospun PCL

In order to observe the behavior of primary cultured cells on electrospun fiber, phase contrast microscopy was used. Phase contrast microscopy converts small phase shifts in the light passing through a transparent specimen into contrast changes making it possible to study the real time cell behavior (proliferation, morphology) using computerized software programs without staining, an advantage particularly important in observing primary (unlabeled) human cells.

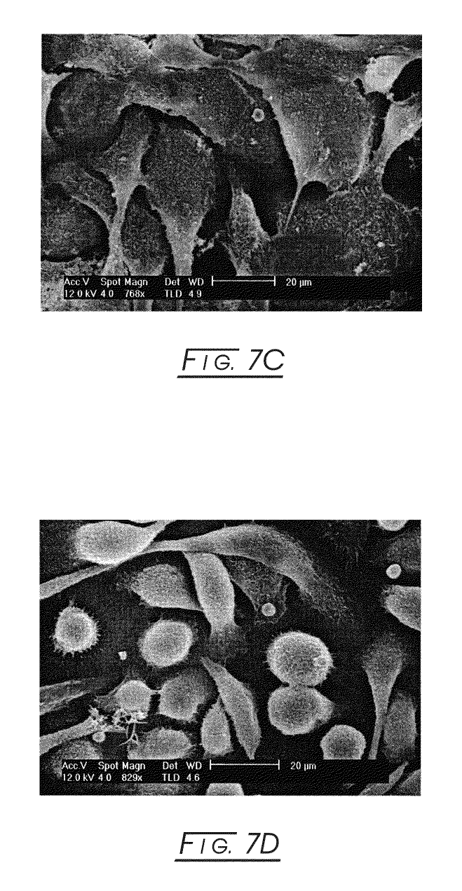

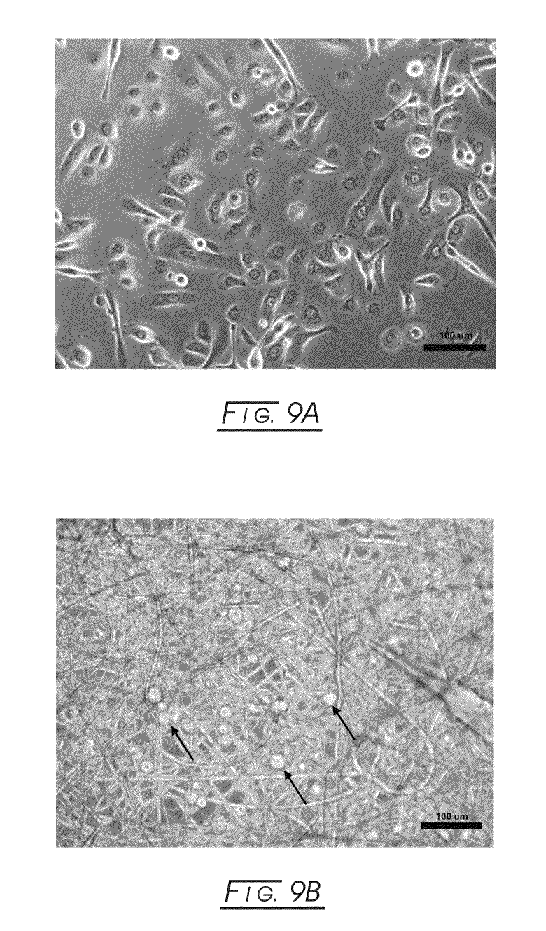

Human normal breast epithelial cells were seeded in a 6-well TCPS (FIG. 9A) and on a 40 μm thick (approximately 30 seconds of electrospinning) 3-D PCL nano-fiber (FIG. 9B) for 48 hours. This particular thickness of electrospun structure was chosen makes it easier to observe seeded cells using inverted phase contrast microscopy. On the nanofiber human breast epithelial cells assume rounded shapes that are quite distinct from the fibroblastic morphology of breast epithelial cells spread out on TCPS. Human breast epithelial cells cultured on 50 and 60 μm thickness electrospun PCL (not shown) are not clearly visible in inverted phase contrast microscopy. Further decreases in the electrospun fiber thickness to less than 40 μm while retaining optimal porosity for different human normal and cancerous breast cells is clearly needed.

### Four Layer IMEMS Technology

Utilizing precisely the same four-layer arrangement of normal (non-cancerous) human breast epithelial cells, stromal cells, breast pre-adipocytes and floating fat globules (pictured in FIG. 1) all from the same human patient, we conducted a preliminary evaluation of the four-layer IMEMS concept. Once fabricated (see FIG. 1), the IMEMS chamber was utilized according to the following: after trypsinization, cells were suspended in co-cultured medium DMEM/F12 supplemented with 5 mg/ml hydorcortisone, 5 mg/ml Insulin, 100 ng/ml cholera toxin, 1% antibiotics and 10% FBS. The primary cultured human normal breast epithelial cells (PCHNBECs) (1.2*104 cells/cm²) were seeded in the lower IMEMS layer and primary cultured human normal breast pre-adipocytes (PCHNBPAs) (6,000 cells/cm²) were seeded in the upper layers. Primary cultured human normal breast stroma cells (PCHNBSCs) (6,000 cells/cm²) were seeded in middle layer via injection using a 1 ml syringe. Cells were incubated in 37° C., 24 hours. RNA extraction used TRIzol reagent.

Five important genes relevant to etiological processes of human breast tumorigenesis were examined using real time PCR. CYP7B1 and CYP19 A1 are aromatase enzymes responsible for conversion of adrenal DHEA and ovarian androgens to estrogens, respectively. PTPγ is an estrogen-regulated human breast cancer suppressor gene discovered in our laboratory (137). MMP3 is a gene regulating the breast cell motility relevant to the process of invasion and metastasis. Cyclin D1 is a cell cycle regulating gene. The mRNA expression levels of all 5 genes, CYP7B1, CYP19 A1, PTPγ, MMP3 and Cyclin D1 detected from the human normal breast epithelial cells cultured for 24 hours in our IMEMS model were 1.2, 5.1, 6.1, 45, and 340 fold higher than in breast epithelial cells cultured on TCPS. Our experimental data was the first to demonstrate that the functionality of gene expression in the human normal breast epithelial cells cultured in IMEMS models are distinct from the same breast cell type cultured on TCPS.

### IMEMS in Controlling Stem/Progenitor Cell Behavior

Methodologies to understand the role of stem and progenitor cells with a pre-neoplastic microenvironment are determined both by introduced dietary components and the physical and biochemical surroundings. Stem/progenitor cells isolated from human normal breast tissues (patient 1081703A1, 29 years old, white) were cultured in the form of mammospheres according to the following technique. Human breast reduction normal (˜220 gram) and cancer tissues (˜0.5-1 gram) were obtained through the Tissue Procurement Program in the Comprehensive Cancer Center at The Ohio State University. Tissues were dissociated overnight on a rotary shaker at 37° C. After dissociation, the dissociated tissue was centrifuged for 5 min, 700 rpm in 50 ml centrifuged tubes and the pellets, which was highly enriched with epithelial organoids, was washed for several times with PBS with 2% Fetal Bovine Serum (FBS) and centrifuged at 1,200 rpm in 50 ml centrifuged tubes after each washing. 1-5 ml of pre-warmed trypsin-EDTA was added to the organoids pellet and was pipette with P1000 for 3 minutes, and then 10 ml of cold PBS with 2% FBS was added and centrifuged at 1,200 rpm for 5 min. After centrifugation, supernatant was removed, and 2-4 ml of pre-warmed dispase and 200-400 μl of 1 mg/ml DNAse 1 was added and pipetted for 1 min. 10 ml of cold PBS with 2% FBS was added and the cell suspension was filtered through a 40-μm cell strainer to obtain a single cell suspension. Cells were cultured in suspended in MEBM supplemented with 1× B27, 20 ng/ml EGF, 1 ng/ml Hydrocortisone, 20 mg/ml Getamycin, 5 mg/ml Insulin, 100 mM 2-mercaptoethanol and 1% antibiotic-antimycotic (100 units/ml penicillin G sodium, 100 mg/ml streptomycin sulfate and 0.25 mg/ml amphotericin B) in a 37° C. humidified incubator (5% CO₂: 95% air).

SEMs were taken of mammospheres seeded on the bio-mimetic 3-D PCL nano-fibers (FIG. 10A) versus on TCPS (FIG. 10B) for 48 hours. Total RNA was isolated from cultured stem/progenitor cells for real time PCR to detect cyclin D1, MMP3, ER α, and PTPγ genes. It is clear from the data illustrated in FIG. 10C that the gene expression of cyclin D1, ERα, and PTPγ in stem/progenitor cells cultured on TCPS are higher than the same genes of stem/progenitor cells cultured on bio-mimetic 3-D PCL nanofibers. However MMP3, a matrix metalloproteinase involved in the breakdown of extracellular matrix during embryonic development, was expressed at significantly higher levels in stem/progenitor cells cultured on the electrospun PCL.

### Example 3

### Aligned Fiber in MultiWell Plates

Random Fiber Preparation

An 18 wt-% solution of poly(ε-caprolactone) (Sigma-Aldrich, Inc. Mw=65,000) in acetone (Mallinckrodt Chemicals) was prepared by heating acetone to 50° C. followed by continuous stirring to dissolve the PCL. After cooling to room temperature, the solution was placed in a 60 cc syringe with a 20 gauge blunt tip needle and electrospun using a high voltage DC power supply (Glassman) set to 24 kV, a 20 cm tip-to-substrate distance and a 16 mL/hr flow rate. A 3×3″ (7.6×7.6 cm) sheet approximately 100 μm in thickness was deposited onto aluminum foil in 2 minutes. The PCL sheets were then placed in a vacuum overnight to ensure removal of residual acetone. High resolution ESI analysis (Esquire) was used to establish that the resulting acetone content is beneath our ability to detect it (less than 10 ppm) (138). Scanning electron microscopy (SEM) was used to show that, as-deposited, the fibers form a random array (FIG. 11).

### Aligned Fiber Preparation

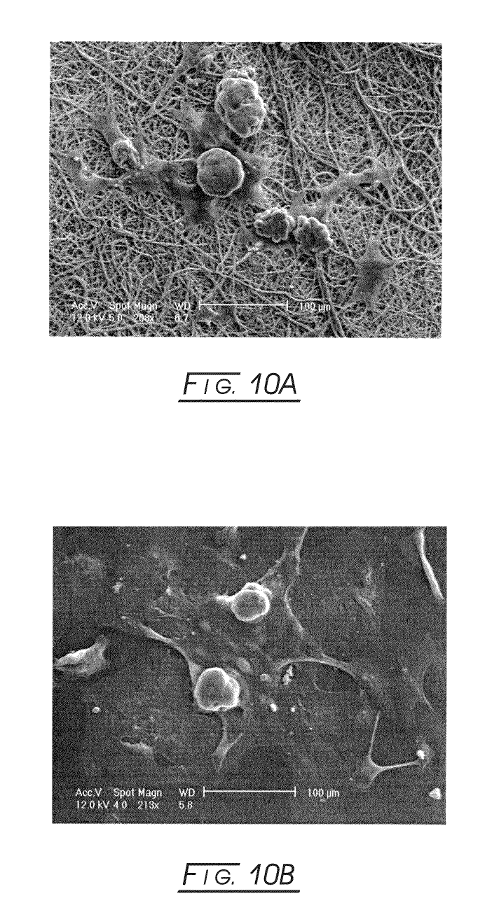

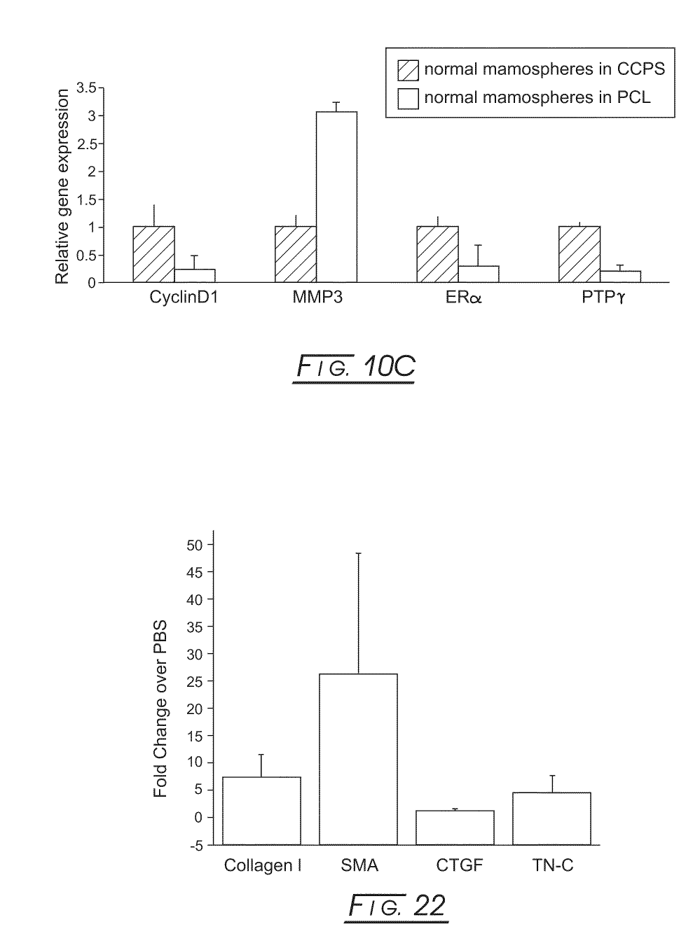

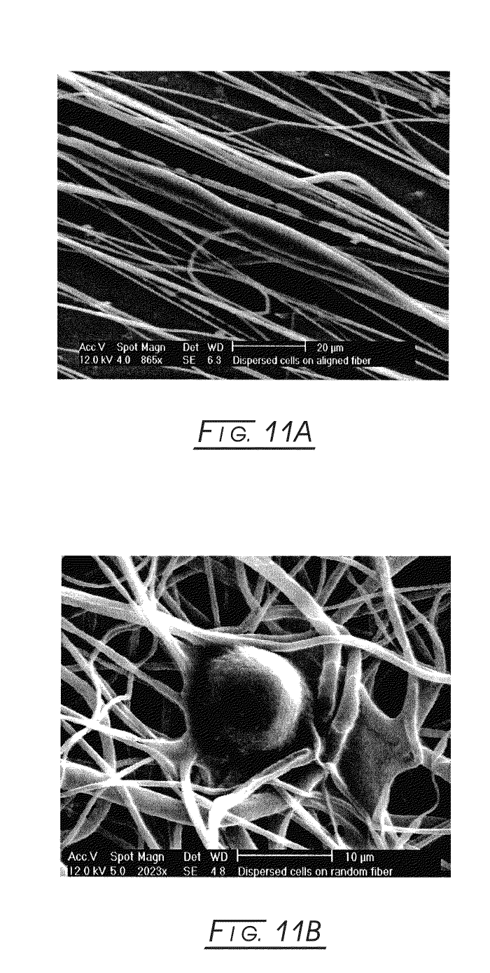

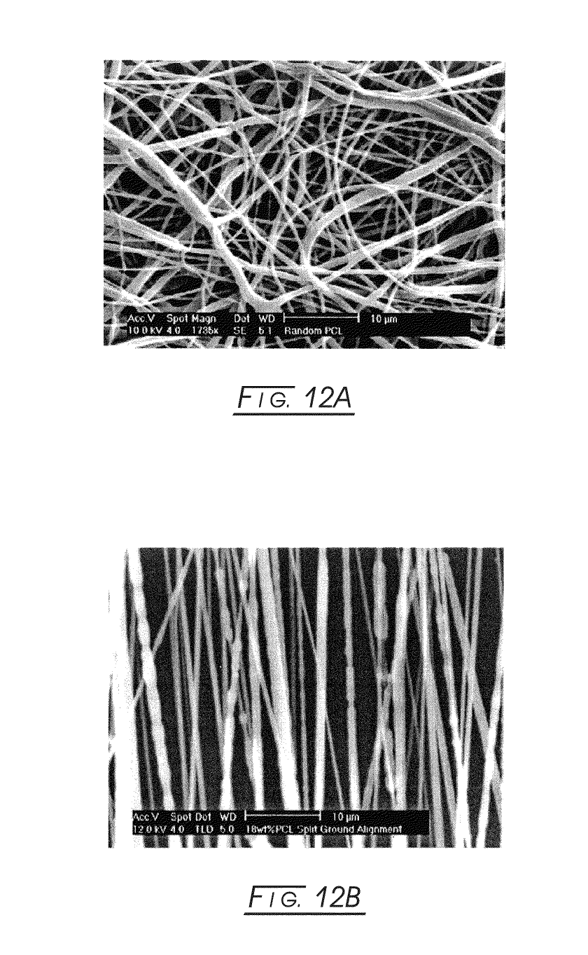

Alignment of the random structure produced by ‘normal’ electrospinning has utilized a variety of techniques (139-142) but the requirements for actively exploring cell migration called for enhanced alignment efficiency combined with levels of production sufficient to match the statistical requirements of cell culture. A method known as the “split ground” (143-147) technique in which fiber deposition rapidly alternates between two separated grounding plates was found to provide relatively efficient alignment (FIG. 12). The same polymer solution used to produce random fiber was electrospun using 10 kV, a horizontal tip-to-substrate distance of 20 cm, and a flow rate of 3 mL/hr. Prior to spinning, fluorescein isothiocynate isomer I (FITC, Fluka BioChemika) was added to cooled solution at 10 mg/mL of polymer solution while stirring continuously. During electrospinning, fibers were deposited for one minute onto a glass disc with two distinct grounds having a 5 mm separation to a thickness of approximately 50 μm. These aligned electrospun samples were again placed in a vacuum overnight, a process proven to ensure removal of residual acetone (138). The samples were then sealed in zip-lock polyethylene bags and the bags submerged in a 45° C. water bath for 10 minutes. This thermal exposure acted as a stress anneal for the aligned fibers and prevented fiber wrinkling during cell culture.

### Dispersed Glioma Culture and Analysis of Migration:

Cell Culture—The human primary glioma cells X12 have been previously described (148) and were routinely maintained in the flanks of immunodeficient (nude) mice. To provide a stable fluorescent label, a suspension of 100,000 X12 cells was infected with 3.4×106 p.f.u. of a lentivirus carrying a red fluorescent protein (RFP) fused to the nuclear protein histone-2B (vector pLenti-H2B-RFP, kindly provided by Dr. Yoshinaga Saeki, OSU). After transduction, cells were re-introduced in host mice and grown until further processing. The human glioma cell line U251 was routinely cultured in Dulbecco's modified Eagle's Medium (DMEM) containing 10% fetal bovine serum, 50 UI/ml penicillin and 50″g/ml streptomycin. These cells were stably transduced using both a cytoplasmic-GFP and a nuclear-RFP lentiviral vectors using standard protocols.

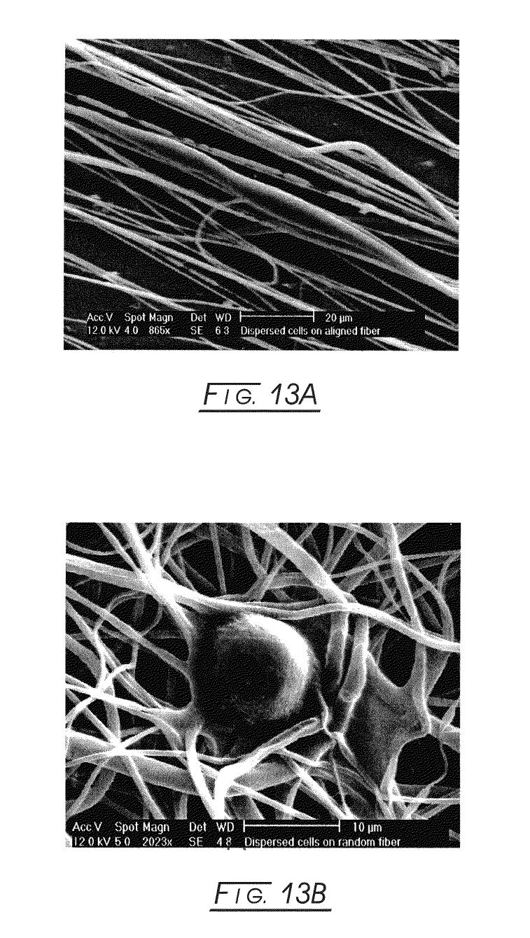

For initial migration studies, X12 or U251 transduced cells were dissociated and seeded on nanofiber substrates at an initial density of ˜7.104 cells/ml (FIG. 13). The cells were dispersed homogeneously on 12 mm diameter discs of random or aligned nanofibers (FIGS. 14A and 14B) that had been attached to the bottom of 35 mm culture dishes, and subsequently tracked over 24 hours using time-lapse microscopy using a confocal microscope (Zeiss LSM 510) fitted with a culture chamber to provide normal temperature and humidity conditions. The cells were identified both by GFP (U251) and RFP (U251 and X12) fluorescence and a 50-μm thick Z-stack was captured every 10 minutes.

### Migration Analysis

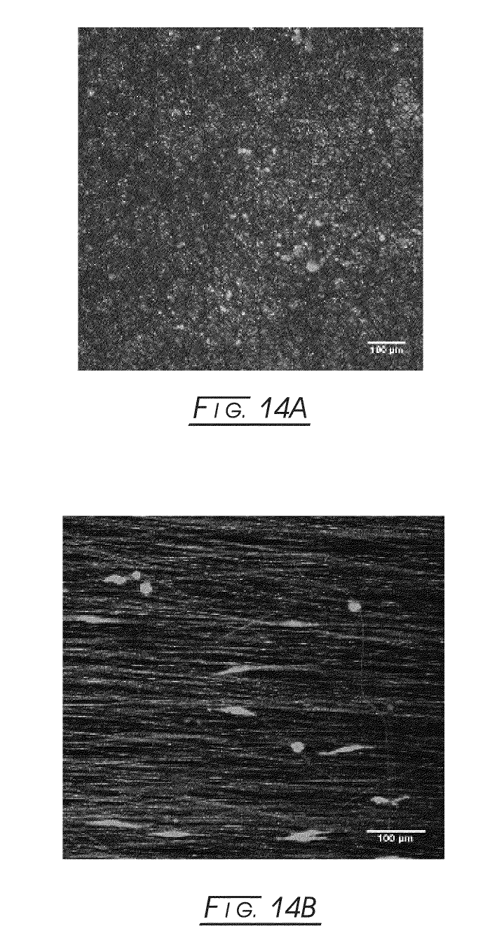

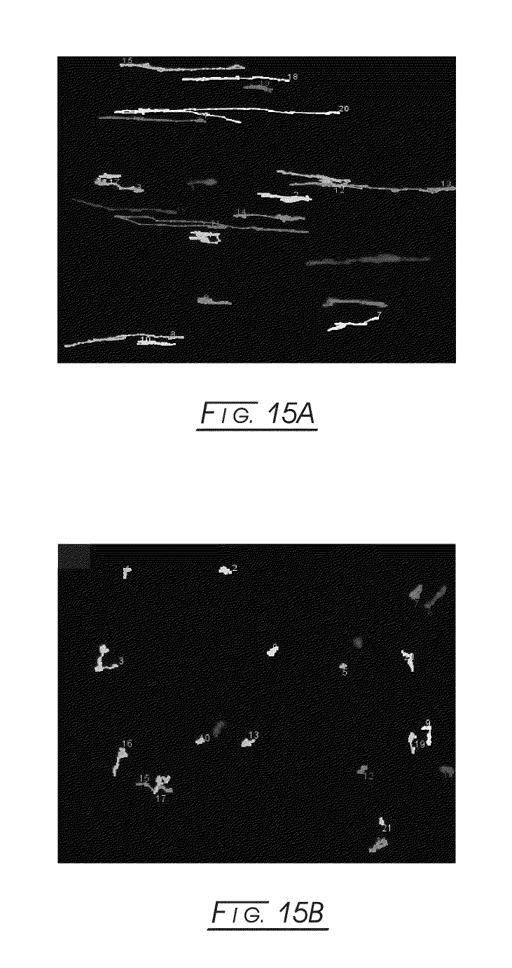

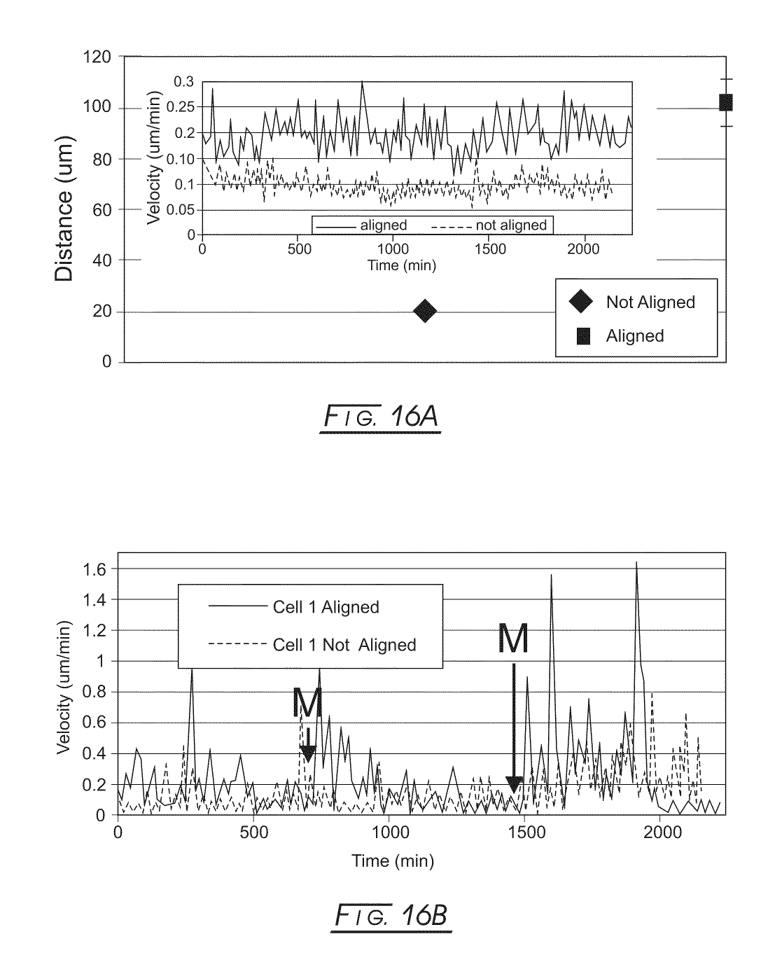

Time-lapse confocal images were processed using ImageJ software to produce a maximal-intensity Z-projection at each captured time point. These images were then concatenated and their illumination normalized, to produce movies that were further analyzed by particle-tracking analysis. Using ImageJ, individual cells were manually identified and tracked throughout the entire duration (˜24 hr) of the experiment (FIGS. 15A and 15B). Total distance traveled, average and individual cell velocities were then quantified and plotted over time (FIGS. 16A and 16B).

Our results demonstrated significant differences in cell migration onto differently oriented nanofibers. In particular, cells seeded on random fibers displayed little progress in any particular direction and remained globular, often contacting multiple fibers and oscillating around the fiber crossings. In marked contrast, cells seeded on aligned fibers exhibited very elongated morphologies (FIG. 13) and had a decisive motion along the fiber axis, characterized by bursts of movement followed by brief pauses to divide. The average velocity of cells on aligned fibers was ˜5 fold higher than that of cells on random fibers, which interestingly matches the ratio of velocities observed for glioma cells migrating in vivo on white versus grey matter (25, 149, 150). It is tempting to speculate whether, even without biomolecular coatings, the scaffolds tested here might have been reproducing the micro and nanoscale alignment of molecular components in the brain.

Time-lapse analysis of these cells provided a quantitative advantage compared to standard, 2D motility assays that usually measure migration as an average of population dispersion between two discrete time points (101). Individual cell tracking showed that actual motion is complex and depends on cell cycle and the local environment. We estimated a mean cell velocity of 4.2″m/h (=3 mm/month) on aligned fibers and 0.8 μm/h (=0.6 mm/month) on random fibers. The result on aligned fibers is lower than what has been measured in 2D assays (12.5 μm/h, (134)) but matches the 4.8 μm/h observed in experimental models in vivo (48) representative of high-grade gliomas (135). The results on random fibers were consistent with observations of gliomas developing in deep grey matter, where white matter tracts are largely absent (136). Thus, these results suggests that the motility of glioma cells on fibrous substrates may be more representative of true glioma migration than what has been observed in previous in vitro models.

### Neurosphere Glioma Culture and Analysis of Migration:

Cell culture—Fresh biopsy samples from high-grade gliomas were dissociated and cultured as previously described to isolate the population of glioma stem cells (151). Cells were maintained in Neurobasal culture medium (Invitrogen) supplemented with 50 ng/ml EGF, 50 ng/ml bFGF, 10 ng/ml LIF and B27 supplement (Invitrogen), and grown in suspension as tumor aggregates known as “neurospheres”. Neurospheres were dissociated by trypsinization and the isolated cells were stably transduced for GFP expression with the lentiviral vector pCDH1-MCSEF1-coGFP (System Biosciences), following the manufacturer's recommendations. GFP-expressing neurospheres were seeded on discs of random or aligned PCL fibers (FIG. 17) and followed by timelapse confocal microscopy, as described above.

### Migration Analysis

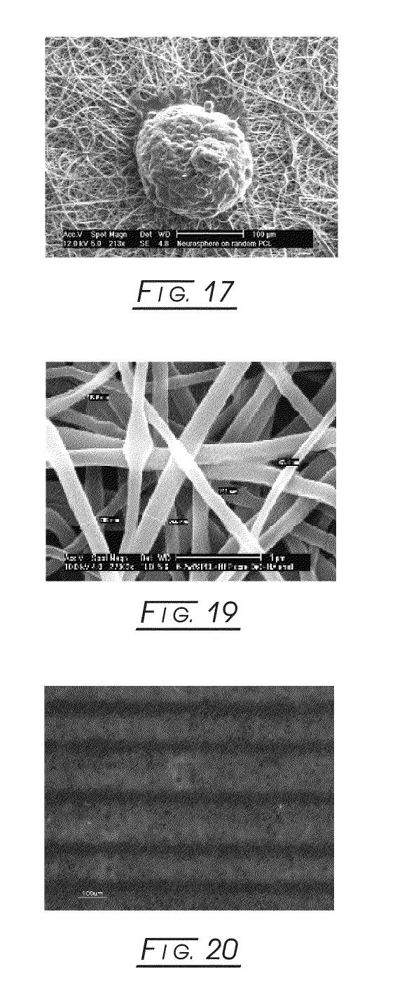

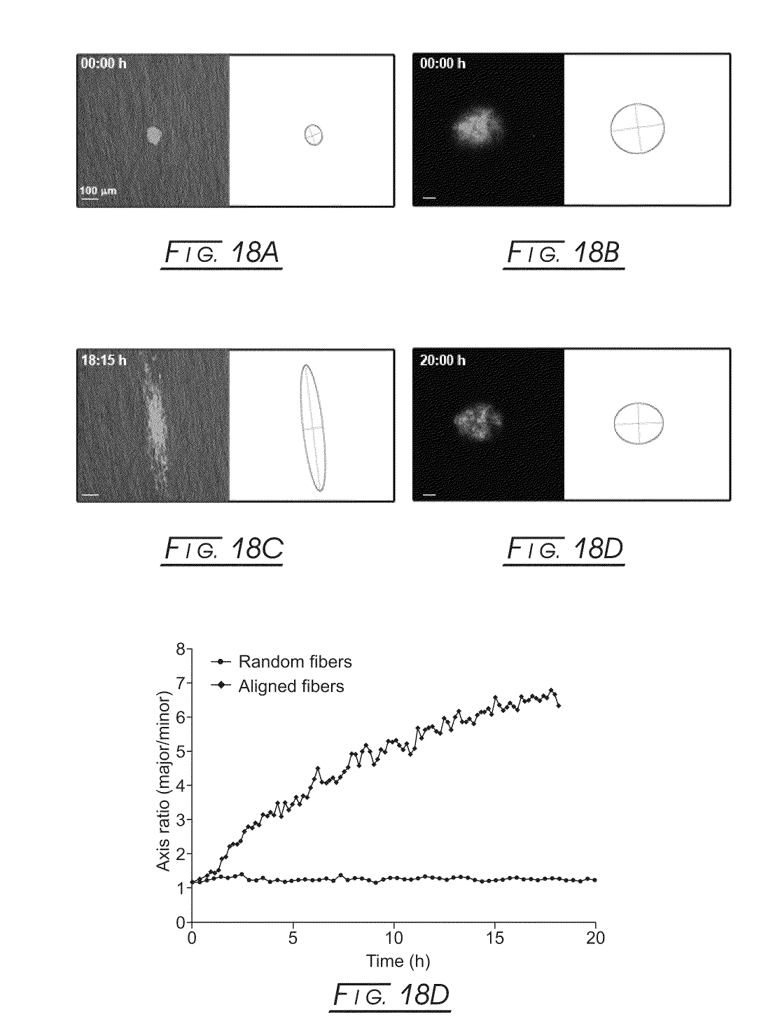

Time-lapse confocal images were processed using ImageJ to produce movies of cell migration, as described above. The core mass of the neurospheres and their detached cells were analyzed as a population of scattered particles using principal component analysis (Viapiano, unpublished). The major and minor axes of cell migration were calculated as eigenvectors of the covariance matrix of particle dispersion, and were further scaled to cover >95% of the original distribution of cells. Essentially, the results showed that neurospheres seeded on random fibers did not spread and instead retained their original shape over time (FIG. 18). This absence of cell detachment from the core neurosphere suggested that cell-substrate adhesion on random fibers might have not been enough to overcome the cellcell adhesions that maintain the spheres. In stark contrast, cells from neurospheres seeded on aligned fibers detached from the core mass and migrated extensively along the fibers, dispersing in this direction more than 6-fold over the orthogonal direction. Interestingly, these cells exhibited almost no migration perpendicular to the fiber alignment.

Taken together, these results not only demonstrate the influence of substrate alignment on glioma cell behavior but also suggest that the different behaviors could be supported by different molecular mediators (e.g., specific adhesion complexes on aligned fibers that the cells fail to form on random fibers).

### Microarray Analysis of Glioma Cells Seeded on Aligned and Random Fibers:

### Cell Culture

Human U251 glioma cells were cultured as described above, dispersed and seeded on random or aligned fibers at an initial density of 1×105 cells/ml. The cells were allowed to settle on the fibers and migrate for 48 to 72 hours, after which they were processed and collected for total RNA analysis by microarrays as described below.

### Microarray Analysis

To analyze transcript expression, total RNA was extracted using Trizol (Invitrogen) and its quality was verified by capillary electrophoresis (Bioanalyzer 2100, Agilent). RNA samples were processed for hybridization to U133Plus 2.0 genechips (Affimetrix), covering the complete human genome. Microarrays were performed in duplicate for each experimental condition (substrate×total time of migration). All procedures, from RNA hybridization and image scanning to data filtering and analysis were performed at the Microarray Core Facility of the OSU Comprehensive Cancer Center. Post-hoc analysis was focused on the transcripts consistently up and down-regulated in the cells migrating on aligned fibers for 48 and 72 hours, compared to cells migrating on random fibers (see Table I for upregulated transcripts).

TABLE 1

| Gene ID | Description | Aligned/Random | Comments |
|---|---|---|---|
| KLF2 | Kruppel-like factor 2 (lung) | 2.658 | leukocyte |
| | | | mobilization |
| PPP1R15A | protein phosphatase 1, | 2.088 | cell signaling |
| | regulatory (inhibitor) subunit 15A | | |
| PPP1R15A | protein phosphatase 1, | 2.086 | cell signaling |
| | regulatory (inhibitor) subunit | | |
| | 15A | | |
| THSD4 | thrombospondin, type I, | 2.078 | anti-angiogenesis |
| | domain containing 4 | | |
| CD55 | Decay accelerating factor for | 2.036 | immune evasion |
| | complement (Cromer blood | | |
| | group) | | |
| CD55 | Decay accelerating factor for | 2.036 | immune evasion |
| | complement (Cromer blood group) | | |
| SPHK1 | sphingosine kinase 1 | 1.998 | glioma cell motility |
| | | | (191) |
| CXCL3 | chemokine (C—X—C motif) | 1.981 | paracrine signaling |
| | ligand 3 | | |
| CXCL2 | chemokine (C—X—C motif) | 1.948 | paracrine signaling |
| | ligand 2 | | |
| HBEGF | heparin-binding EGF-like | 1.942 | ECM-binding |
| | growth factor | | cytokine |
| CD55 | Decay accelerating factor for | 1.939 | immune evasion |
| | complement (Cromer blood | | |
| | group) | | |
| MICAL2 | microtubule associated | 1.753 | cytoskeletal |
| | protein | | regulation |
| MICAL2 | microtubule associated | 1.691 | cytoskeletal |
| | protein | | regulation |
| EPHB2 | EPH receptor B2 | 1.658 | neural cell motility |
| C9orf150 | chromosome 9 open reading | 1.628 | — |
| | frame 150 | | |
| SLC38A1 | solute carrier family 38, | 1.616 | aminoacid |
| | member 1 | | transport |
| LTBP2 | latent transforming growth | 1.585 | TGF signaling |
| | factor beta binding protein 2 | | |
| FRMD6 | FERM domain containing 6 | 1.576 | actin-binding, |
| | | | motility |
| STX1A | syntaxin 1A (brain) | 1.548 | neural cell |
| | | | connectivity |
| GFPT2 | glutamine-fructose-6- | 1.521 | oxidative |
| | phosphate transaminase 2 | | metabolism |
| PIK3CD | phosphoinositide-3-kinase, | 1.519 | leukocyte homing |
| | catalytic, delta polypeptide | | |
| IRAK2 | interleukin-1 receptor- | 1.514 | cytokine receptor |
| | associated kinase 2 | | |

Table I: List of transcripts specifically upregulated (>1.5-fold, p < 0.005) in U251 cells seeded on aligned versus random PCL fibers. Hybridization signal intensities were filtered and normalized to the average values of cells on random fibers. Known and potential pro-migratory genes were bolded.

### Biochemically Modified Nanofibers:

To enhance the resemblance of these nanoscale scaffolds to the in vivo environment, two techniques were employed: core-shell electrospinning and ink-jet printing. Examples are given below.

### Core-Shell Fibers:

Dil cyanine dye (dialkylcarbocyanine, Invitrogen) was added at a ratio of 1:100 to a 1 mg/mL solution of hyaluronic acid (Calbiochem) in DI water. The 18 wt-% PCL in acetone solution was prepared as before (FIG. 19). Nanoscale core-shell fibers were then prepared (FIG. 19) using a 22 gauge hypodermic needle (Integrated Dispensing Solutions) inserted through a 16 gauge hypodermic T-junction (Small Parts, Inc) to create the required two concentric blunt needle openings (148). A Swagelok stainless steel union was used to hold the needles in place and ensure the ends of the needles were flush with each other. One syringe (BD Luer-Lok tip) was filled with the polymer solution for the ‘core,’ connected to the 22-gauge needle and set to a 2-mL/hr flow rate using a syringe pump. Another identical syringe was connected via an extension to the T-junction, filled with the desired ‘shell’ solution and set to a flow rate of 1 mL/hr using a separate syringe pump. A high voltage power source (Glassman High Voltage, Inc.) was connected to the concentric needle structure and set to +28 kV for the PCL+HFP polymer solution or +25 kV for the PCL+acetone polymer solution with a tip-to-substrate distance of 20 cm.

A split ground technique using the same solutions and voltages was utilized to create aligned fibers spanning the two electrodes separated by 5 mm on a glass cover slip. Additionally, more aligned fibers were produced by depositing onto a rotating mandrel with a linear velocity of 18.3 m/s. High pressure CO₂ was used to infuse/bond the shell to the core using a 30 minute exposure at 900 psi.

### Ink-Jet Printing:

Rat brain myelin was purified by differential centrifugation using the original method of Norton and Poduslo (152) and further purified by osmotic shock and additional isopycnic sedimentation as previously described (153). Highly purified myelin was resuspended in 20 mM TrisHCl, pH 7.4 (˜0.1 mg/ml total protein), aliquoted and diluted at a ratio of 1:9 in PBS. Dil cyanine dye was then added at a volumetric dye-solution ratio of 1:100. This solution was then printed onto the substrate using an industrial grade ink jet printer (Jetlab II, Microfab Technologies, Inc. Plano, Tex.) employing a glass capillary tip with having an orifice diameter of 50 μm. A drop frequency of 180 Hz was used in combination with a head speed of 5 mm/s. A custom-made program script was used to produce the printed pattern seen in FIG. 20.

In summary, these preliminary results show that: a) nanoscaled physical structures exert dominant effects on glioma cell motility; b) substrate topography influences gene expression that may directly relate to tumor migration; and c) we have a variety of tools to optimize these substrates in ways that will enhance their resemblance to the in vivo environment.

### Example 4

In this example, electrospinning utilizes polycaprolactone (PCL), a synthetic fiber that has been demonstrated to serve as an optimal scaffold for cells to adhere to and move freely as they divide and differentiate [154, 155-157]. Preliminary data uses PCL scaffolds to which bleomycin-treated lung extracts have been coated onto the fibers themselves. These fibers, which then contain components of this fibrotic microenvironment, are placed onto a cell culture dish. Any cell of interest can be plated on the coated matrix, and the morphological interaction of the cells with this scaffold observed via scanning electron microscopy (SEM). The cells can also be removed for molecular analyses. This system is ideal in that it allows us to test our hypothesis that the fibrotic microenvironment is responsible for the differentiation of bone marrow cells into (myo)fibroblasts in vivo, by way of an ex vivo model. In the long-term, this system is particularly powerful in that conditions of the matrix can be altered, such as cytokine addition or subtraction of specific growth factors or proteins to more realistically determine which of these specific factors are responsible for myofibroblast differentiation in the chemically complex microenvironment. This technology is highly translational, as we would be able to use tissues from patients to create a matrix environment representing a disease of interest, and then plate human cells onto the matrices as a diagnostic measure. This technology can also be used in any disease or organ, not only the lung, rendering it a powerful platform for studying various disease states.

### Results

### Biological Effects of Lung Extract Coatings on PCL Nanofiber

To determine the feasibility of our first aim, nanofiber matrices were spun [155, 156] and coated with bleomycin-treated mouse lung extract [158] using a TC-100 Desktop spin coater (MTI Corporation). Electrospun fiber sheets were cut into 22-mm diameter discs and glued to glass coverslips using a biocompatible silicone glue (Part #40076 Applied Silicone Corporation). The fiber discs were then placed on the spin coater at 1,000 RPM and pre-wetted with 75 μL of ethanol and then coated with 75 μL of extract. After air-drying, the extract was cross-linked onto the fibers as described elsewhere [159]. For comparison purposes, other matrices were spin coated with PBS-treated lung extract, and placed in 12-well cell culture dishes. Bone marrow from wildtype FVB/N mice was extracted and the white blood cells were isolated and plated in DMEM with 2% FBS. Cells were viewed with SEM after 2, 4, 8, and 14 days. As shown in FIGS. 21A and 21B, bone marrow cells plated on matrices coated with bleomycin-treated lung extract began to secrete matrix materials and clump together, whereas bone marrow cells plated on matrices coated with PBS-treated lung extract did not.

Next, RNA was isolated from wild-type mouse bone marrow cells after they were plated on nanofiber matrices coated with lung extracts from either PBS or bleomycin treated mice. As shown in FIG. 22, type-I collagen, alpha smooth muscle actin, and tenascin-C expression were increased in cells plated on nanofibers that were coated with bleomycin treated mouse lung extracts compared to cells plated on nanofibers coated with lung extracts from mice treated with PBS. This experiment provided significant indications that an appropriately-coated nanofiber matrix could give rise to fibrotic responses in arriving BMSCs.

### Biological Effects of Nanofiber Modulus

To further establish the role of matrix stiffness independent of matrix interactions with plated cells, we next tested nanofiber matrices having different moduli were spun from synthetic polymers. Polyethersulfone (PES) has a modulus (385,000 psi) ˜28.5 times greater than the previously (FIGS. 21A, 21B, and 22) utilized PCL (13,500 psi). This provides a dramatically different modulus appropriate to determining the effects of modulus (if any) on cellular response. Finally, we included a “core-shell” fiber [160-163] consisting of a thin ‘shell’ of PCL on a ‘core’ of PES. This provides an elegant means of retaining the PCL surface chemistry while increasing the overall fiber modulus from 7.1 MPa (pure PCL) to 30.6 MPa, an increase of more than 4 times [164].

A 8 wt-% solution of polyethersulfone (PES) (Goodfellow, Cambridge, UK) in 1,1,1,3,3,3-Hexafluoro-2-propanol (HFIP) (Sigma-Aldrich) was prepared by continuous stirring. The solution was then placed in a 60-cc syringe with a 20-gauge blunt tip needle and electrospun using a high voltage DC power supply (Glassman High Voltage, Inc.) set to +23 kV, a 20 cm tip-to-substrate distance and a 5 ml/hr flow rate. A 3×3″ (7.6×7.6 cm) sheet approximately 0.2 mm in thickness was deposited onto aluminum foil. PES fiber sheets were then placed in a vacuum overnight (to ensure removal of residual HFIP [157]) and cut into 22 mm diameter discs and glued to glass coverslips for cell culture as before (see FIG. 1).

Core-shell fibers were prepared by using a 22-gauge hypodermic needle (Integrated Dispensing Solutions Agoura Hills, Calif.) inserted through a 16-gauge hypodermic T-junction (Small Parts, Inc. Miramar, Fla.) to create two concentric blunt needle openings. A Swagelok stainless steel union was used to hold the needles in place and ensure the ends of the needles were flush with each other. One syringe (BD Luer-Lok tip) was filled with the polymer solution for the core, PES+HFIP, connected to the 22-gauge needle and set to a 2 mL/hr flow rate using a syringe pump. Another identical syringe was connected via an extension to the T-junction, filled with the shell material, PCL+HFIP, and set to a flow rate of 2 mL/hr using another syringe pump. A high voltage power source (Glassman High Voltage, Inc.) was connected to the concentric needle structure and set to +30 kV for the PES+HFIP polymer solution core with a shell of PCL+HFIP and a tip-to-substrate distance of 20 cm. PCL/PES core shell fiber sheets were then placed in a vacuum overnight (to ensure removal of residual HFIP [38]) and cut into 22 mm diameter discs and glued to glass cover slips for cell culture as before (see FIG. 1).

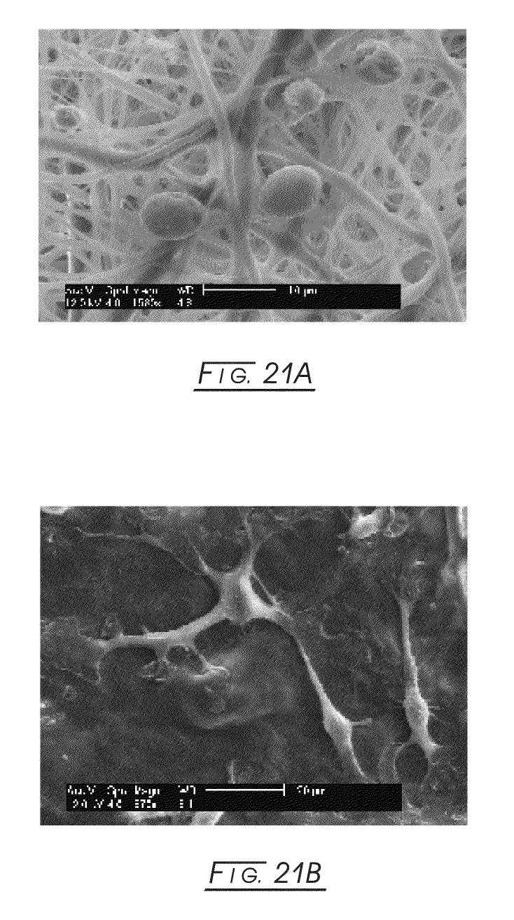

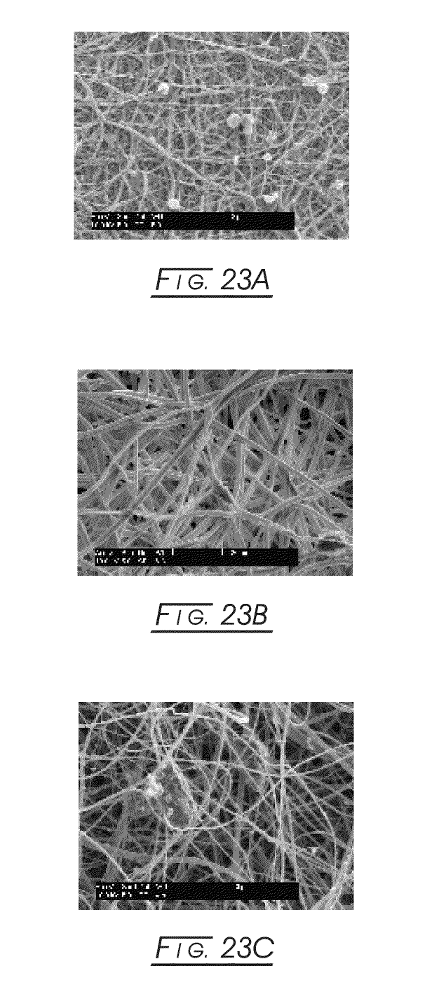

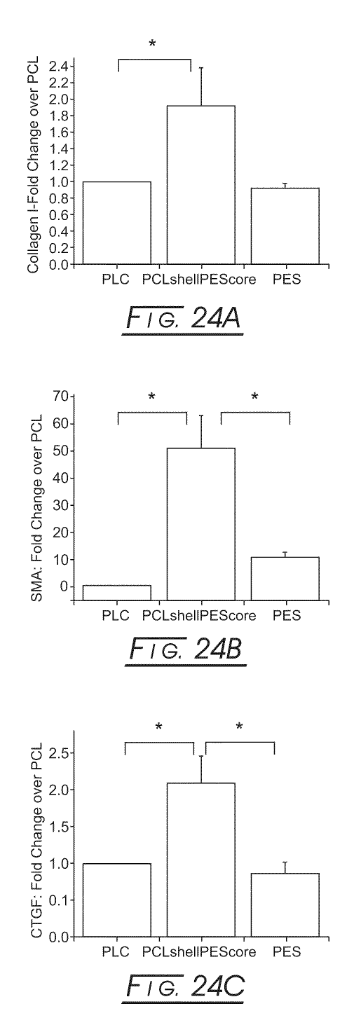

FIGS. 23A, 23B, and 23C shows that bone marrow cells plated on uncoated PCL (modulus used in coating studies), PCL/PES (higher modulus with PCL coating) and PES (higher modulus with change in coating) produced similar cell morphologies on all three nanofiber compositions. RNA isolated from wild-type mouse bone marrow cells cultured for 8 days on these nanofiber matrices showed substantially different behaviors, however. As shown in FIGS. 24A, 24B, and 24C, type-I collagen, alpha smooth muscle actin and connective tissue growth factor were increased in cells plated on the PCL/PES core-shell nanofibers. Given that this nanofiber composition has the same surface chemistry as PCL, these results suggest that nanofiber modulus plays a significant role in conditioning the fibrotic response of BM-derived cells.

### Summary

Taken together, these initial observations suggest that BM cells exposed to a fibrotic lung microenvironment develop a more fibroblast/myofibroblast phenotype than cells exposed to a ‘normal’ lung microenvironment. In addition, the modulus of the underlying nanomatrix plays a significant role in the acuteness of this response. Further work to establish the clinical potential of this model involves (a) a deeper understanding of the biochemical makeup of the lung extract coating, (b) knowledge of substrate properties/preparation that more efficiently drive differentiation down fibrotic pathways, (c) establishing the cellular and molecular changes in BMSCs challenged by different substrates and biochemical stimuli and (d) the use of appropriate pharmacological inhibitors to identify drugs and strategies with the potential to reduce fibrotic responses.

Unless otherwise defined, all technical and scientific terms used herein have the same meaning as commonly understood by one of ordinary skill in the art to which this invention pertains, methods, and examples are illustrative only and not intended to be limiting. While the device and process have been described with reference to various embodiments, those skilled in the art will understand that various changes may be made and equivalents may be substituted for elements thereof without departing from the scope and essence of the disclosure. In addition, many modifications may be made to adapt a particular situation or material to the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the metric system and all amounts and percentages are by weight, unless otherwise expressly indicated. Also, all citations referred herein are expressly incorporated herein by reference.

### REFERENCES

1. Wykosky J, Palma E, Gibo D M, Ringler S, Turner C P, Debinski W 2008 Soluble monomeric EphrinA1 is released from tumor cells and is a functional ligand for the EphA2 receptor. Oncogene 27(58):7260-7273.
2. Rocks N, Paulissen G, Quesada-Calvo F, Munaut C, Gonzalez M L A, Gueders M, Hacha J, Gilles C, Foidart J M, Noel A, Cataldo D D 2008 ADAMTS-1 Metalloproteinase Promotes Tumor Development through the Induction of a Stromal Reaction In vivo. Cancer Research 68(22):9541-9550.
3. Lieblein J C, Ball S, Hutzen B, Sasser A K, Lin H J, Huang T H M, Hall B M, Lin JY 2008 STAT3 can be activated through paracrine signaling in breast epithelial cells. Bmc Cancer 8. References Cited Page 85 Principal Investigator/Program Director (Last, first, middle): Johnson, Jed, Kizer
5. Levina V, Su Y Y, Nolen B, Liu X N, Gordin Y, Lee M, Lokshin A, Gorelik E 2008 Chemotherapeutic drugs and human tumor cells cytokine network. International Journal of Cancer 123(9):2031-2040.
6. 22. Luu Y K, Kim K, Hsiao B S, Chu B, Hadjiargyrou M 2003 Development of a nanostructured DNA delivery scaffold via electrospinning of PLGA and PLA-PEG block copolymers. Journal of Controlled Release 89(2):341-353.
7. Reneker D H, Chun 11996 Nanometre diameter fibres of polymer, produced by electrospinning. Nanotechnology 7(3):216-223.
8. Li W J, Tuli R, Huang X X, Laquerriere P, Tuan R S 2005 Multilineage differentiation of human mesenchymal stem cells in a three-dimensional nanofibrous scaffold. Biomaterials 26(25):5158-5166.
9. Fujihara K, Kotaki M, Ramakrishna S 2005 Guided bone regeneration membrane made of polycaprolactone/calcium carbonate composite nano-fibers. Biomaterials 26(19):4139-4147.
10 Kwon I K, Kidoaki S, Matsuda T 2005 Electrospun nano- to microfiber fabrics made of biodegradable copolyesters: structural characteristics, mechanical properties and cell adhesion potential Biomaterials 26(18):3929-3939.
11. Zeng J, Chen X S, Liang Q Z, Xu X L, Jing X B 2004 Enzymatic degradation of poly(L-lactide) and poly(epsilon-caprolactone) electrospun fibers. Macromolecular Bioscience 4(12):1118-1125.
12. Khil M S, Bhattarai S R, Kim H Y, Kim S Z, Lee K H 2005 Novel fabricated matrix via electrospinning for tissue engineering. Journal Of Biomedical Materials Research Part B-Applied Biomaterials 72B(1):117-124.
13 Zhang Y Z, Ouyang H W, Lim C T, Ramakrishna S, Huang Z M 2005 Electrospinning of gelatin fibers and gelatin/PCL composite fibrous scaffolds. Journal Of Biomedical Materials Research Part Applied Biomaterials 72B(1):156-165.
14 Li W J, Tuli R, Okafor C, Derfoul A, Danielson K G, Hall D J, Tuan R S 2005 A three-dimensional nanofibrous scaffold for cartilage tissue engineering using human mesenchymal stem cells. Biomaterials 26(6):599-609.
15. Hsu C M, Shivkumar S 2004 Nano-sized beads and porous fiber constructs of poly(epsilon-caprolactone) produced by electrospinning. Journal Of Materials Science 39(9):3003-3013.
16. Hsu C M, Shivkumar S 2004 N,N-dimethylformamide additions to the solution for the electrospinning of poly(epsilon-caprolactone) nanofibers. Macromolecular Materials And Engineering 289(4):334-340.
17. Shin M, Ishii O, Sueda T, Vacanti J P 2004 Contractile cardiac grafts using a novel nanofibrous mesh. Biomaterials 25(17):3717-3723.
18. Shin M, Yoshimoto H, Vacanti J P 2004 In vivo bone tissue engineering using mesenchymal stem cells on a novel electrospun nanofibrous scaffold. Tissue Engineering 10(1-2):33-41.
19. Li W J, Danielson K G, Alexander P G, Tuan R S 2003 Biological response of chondrocytes cultured in three-dimensional nanofibrous poly(epsilon-caprolactone) scaffolds. Journal Of Biomedical Materials Research Part A 67A(4):1105-1114.
20. Zeng J, Chen X S, Xu X Y, Liang Q Z, Bian X C, Yang L X, Jing X B 2003 Ultrafine fibers electrospun from biodegradable polymers. Journal Of Applied Polymer Science 89(4):1085-1092.
21. Yoshimoto H, Shin Y M, Terai H, Vacanti JP 2003 A biodegradable nanofiber scaffold by electrospinning and its potential for bone tissue engineering. Biomaterials 24(12):2077-2082.
22. Lee K H, Kim H Y, Khil M S, Ra Y M, Lee D R 2003 Characterization of nano-structured poly(epsiloncaprolactone) nonwoven mats via electrospinning. Polymer 44(4):1287-1294.
23. Johnson J, Niehaus A, Nichols S, Lee D, Koepsel J, Anderson D, Lannutti J 2009 Electrospun PCL in Vitro: a Microstructural Basis for Mechanical Property Changes. Journal of Biomaterials Science-Polymer Edition 20(4):467-481.
24. Gaumer J, Prasad A, Lee D, Lannutti J 2009 Source-to-ground Distance and the Mechanical Properties of Electrospun Fiber. Acta Biomaterialia 5(5):1552-1561. References Cited Page 86 Principal Investigator/Program Director (Last, first, middle): Johnson, Jed, Kizer
25. Drilling S, Gaumer J, Lannutti J 2009 Fabrication of burst pressure competent vascular grafts via electrospinning: Effects of microstructure. Journal of Biomedical Materials Research Part A 88A(4):923-934.
26. Duling R R, Dupaix R B, Katsube N, Lannutti J 2008 Mechanical characterization of electrospun polycaprolactone (PCL): a potential scaffold for tissue engineering. Journal of Biomechanical Engineering 130 (1):Article Number: 011006.
27. Martins A, Araujo J V, Reis R L, Neves N M 2007 Electrospun nanostructured scaffolds for tissue engineering applications. Nanomedicine 2(6):929-942.
28. Lannutti J, Reneker D, Ma T, Tomasko D, Farson D 2007 Electrospinning for tissue engineering scaffolds. Materials Science and Engineering: C 27:504-509.
29. Johnson J, Ghosh A, Lannutti J 2007 Microstructure-Property Relationships in a Tissue Engineering Scaffold. Journal Of Applied Polymer Science 104(5):2919-2927.
30. Teo W E, Ramakrishna S 2006 A review on electrospinning design and nanofibre assemblies. Nanotechnology 17(14):R89-R106.
31. Zhang Y Z, Lim C T, Ramakrishna S, Huang Z M 2005 Recent development of polymer nanofibers for biomedical and biotechnological applications. Journal of Materials Science-Materials in Medicine 16(10):933-946.
32. Wang Y K, Yong T, Ramakrishna S 2005 Nanofibres and their influence on cells for tissue regeneration. Australian Journal of Chemistry 58(10):704-712.
33. FDA 1974 Food Drug Cosmetic Law 56, pp 518.94, 172.894.
34. Leblanc M, Russo J, Kudelka A P, Smith J A 2002 An in vitro study of inhibitory activity of gossypol, a cottonseed extract, in human carcinoma cell lines. Pharmacological Research 46(6):551-555.
35. Liang X S, Rogers A J, Webber C L, Ormsby T J, Tiritan M E, Matlin S A, Benz C C 1995 Developing gossypol derivatives with enhanced antitumor activity. Investigational New Drugs 13(3):181-186.
36. Thomas M, Vonhagen V, Moustafa Y, Montmasson M P, Monet J D 1991 EFFECTS OF GOSSYPOL ON THE CELL-CYCLE PHASES IN T-47D HUMAN BREAST-CANCER CELLS. Anticancer Research 11(4):1469-1476.
37. Jaroszewski J W, Kaplan O, Cohen J S 1990 ACTION OF GOSSYPOL AND RHODAMINE 123 ON WILD-TYPE AND MULTIDRUG-RESISTANT MCF-7 HUMAN BREAST-CANCER CELLS—P-31 NUCLEAR-MAGNETIC-RESONANCE AND TOXICITY STUDIES. Cancer Research 50(21):6936-6943.
38. Benz C C, Keniry M A, Ford J M, Townsend A J, Cox F W, Palayoor S, Matlin S A, Hait W N, Cowan K H 1990 BIOCHEMICAL CORRELATES OF THE ANTITUMOR AND ANTIMITOCHONDRIAL PROPERTIES OF GOSSYPOL ENANTIOMERS. Molecular Pharmacology 37(6):840-847.
39. Wu Y W, Chik C L, Knazek R A 1989 AN INVITRO AND INVIVO STUDY OF ANTITUMOR EFFECTS OF GOSSYPOL ON HUMAN SW-13 ADRENOCORTICAL CARCINOMA. Cancer Research 49(14):3754-3758.
40 Band V, Hoffer A P, Band H, Rhinehardt A E, Knapp R C, Matlin S A, Anderson D J 198 ANTIPROLIFERATIVE EFFECT OF GOSSYPOL AND ITS OPTICAL ISOMERS ON HUMA REPRODUCTIVE CANCER CELL-LINES. Gynecologic Oncology 32(3):273-277.
41. Benz C, Keniry M, Goldberg H 1988 SELECTIVE TOXICITY OF GOSSYPOL AGAINST EPITHELIAL TUMORS AND ITS DETECTION BY MAGNETIC-RESONANCE SPECTROSCOPY. Contraception 37(3):221-228.
42. Benz C, Hollander C, Keniry M, James T L, Mitchell M 1987 LACTIC-DEHYDROGENASE ISOZYMES, P-31 MAGNETIC-RESONANCE SPECTROSCOPY, AND INVITRO ANTIMITOCHONDRIAL TUMOR TOXICITY WITH GOSSYPOL AND RHODAMINE-123. Journal of Clinical Investigation 79(2):517-523.
42. Tuszynski G P, Cossu G 1984 DIFFERENTIAL CYTO-TOXIC EFFECT OF GOSSYPOL ON HUMAN-MELANOMA, COLON-CARCINOMA, AND OTHER TISSUE-CULTURE CELL-LINES. Cancer Research 44(2):768-771.
44. Wang Y C, Rao P N 1984 EFFECT OF GOSSYPOL ON DNA-SYNTHESIS AND CELL-CYCLE PROGRESSION OF MAMMALIAN-CELLS INVITRO. Cancer Research 44(1):35-38.
45. Gilbert N E, Oreilly J E, Chang C J G, Lin Y C, Brueggemeier R W 1995 ANTIPROLIFERATIVE ACTIVITY OF GOSSYPOL AND GOSSYPOLONE ON HUMAN BREAST-CANCER CELLS. Life Sciences 57(1):61-67.
46. Hu Y F, Chang C J G, Brueggemeier R W, Lin Y C 1993 GOSSYPOL INHIBITS BASAL AND ESTROGEN-STIMULATED DNA-SYNTHESIS IN HUMAN BREAST-CARCINOMA CELLS. Life Sciences 53(25):PL433-PL438.
47. Liu S L, Kulp S K, Sugimoto Y, Jiang J H, Chang H L, Dowd M K, Wan P, Lin Y C 2002 The (−)-enantiomer of gossypol possesses higher anticancer potency than racemic gossypol in human breast cancer. Anticancer Research 22(1A):33-38.
48. Central Brain Tumor Registry of the United, S. Statistical Report: Primary Brain Tumors in the United States 1998-2002. CBTRUS, 2005.
49. Holland, E. C. Glioblastoma multiforme: the terminator. Proc. Natl. Acad. Sci. U.S.A 97, 6242, 2000.
50. Fathallah-Shaykh, H. M. Darts in the dark cure animal, but not human, brain tumors. Arch. Neurol. 59, 721, 2002.
51. Kleihues, P., Louis, D. N., Scheithauer, B. W., Rorke, L. B., Reifenberger, G., Burger, P. C., and Cavenee, W. K. The WHO classification of tumors of the nervous system. J. Neuropathol. Exp. Neurol. 61, 215, 2002.
52. Tonn, J. C., and Goldbrunner, R. Mechanisms of glioma cell invasion. Acta Neurochir. Suppl 88, 163, 2003.
53. Vuorinen, V., Hinkka, S., Farkkila, M., and Jaaskelainen, J. Debulking or biopsy of malignant glioma in elderly people—a randomised study. Acta Neurochirurgica 145, 5, 2003.
54. Schiffer, D., Cavalla, P., Dutto, A., and Borsotti, L. Cell proliferation and invasion in malignant gliomas. Anticancer Research 17, 61, 1997.
55. Quigley, M. R., and Maroon, J. C. The relationship between survival and the extent of the resection in patients with supratentorial malignant gliomas. Neurosurgery 29, 385, 1991.
56. Furnari F. B., Fenton T., Bachoo R. M., Mukasa A., Stommel J. M, Stegh A. et. al. Malignant astrocytic glioma: genetics, biology, and paths to treatment. Genes Dev. 1, 2683, 2007.
57. Bellail, A. C., Hunter, S. B., Brat, D. J., Tan, C., and Van Meir, E. G. Microregional extracellular matrix heterogeneity in brain modulates glioma cell invasion. Int J Biochem Cell Biol 36, 1046, 2004.
58. Subramanian, A., Harris, A., Piggott, K., Shieff, C., and Bradford, R. Metastasis to and from the central nervous system—the ‘relatively protected site’. Lancet Oncol 3, 498, 2002.
59. Pilkington, G. J. The paradox of neoplastic glial cell invasion of the brain and apparent metastatic failure. Anticancer Res 17, 4103, 1997.
60. Bernstein, J. J., and Woodard, C. A. Glioblastoma cells do not intravasate into blood vessels. Neurosurgery 36, 124, 1995.
61. Georges, P. C., Miller, W. J., Meaney, D. F., Sawyer, E. S., and Janmey, P. A. Matrices with compliance comparable to that of brain tissue select neuronal over glial growth in mixed cortical cultures. Biophys. J. 90, 3012, 2006.
62. Georges, P. C., and Janmey, P. A. Cell type-specific response to growth on soft materials. J. Appl. Physiol 98, 1547, 2005.
63. Lo, C. M., Wang, H. B., Dembo, M., and Wang, Y. L. Cell movement is guided by the rigidity of the substrate. Biophysical Journal 79, 144, 2000.
64. Levicar, N., Nuttall, R. K., and Lah, T. T. Proteases in brain tumour progression. Acta Neurochir. (Wien.) 145, 825, 2003.
65. VanMeter, T. E., Rooprai, H. K., Kibble, M. M., Fillmore, H. L., Broaddus, W. C., and Pilkington, G. J. The role of matrix metalloproteinase genes in glioma invasion: co-dependent and interactive proteolysis. J. Neurooncol. 53, 213, 2001.
66. Ohnishi, T., Matsumura, H., Izumoto, S., Hiraga, S., and Hayakawa, T. A novel model of glioma cell invasion using organotypic brain slice culture. Cancer Res. 58, 2935, 1998.
67. Jung, S., Ackerley, C., Ivanchuk, S., Mondal, S., Becker, L. E., and Rutka, J. T. Tracking the invasiveness of human astrocytoma cells by using green fluorescent protein in an organotypical brain slice model. J Neurosurg 94, 80, 2001.
68. Palfi, S., Swanson, K. R., De, B. S., Chretien, F., Oliveira, R., Gherardi, R. K., Kros, J. M., Peschanski, M., and Christov, C. Correlation of in vitro infiltration with glioma histological type in organotypic brain slices. Br. J. Cancer 91, 745, 2004.
69. Viapiano, M. S., Hockfield, S., and Matthews, R. T. BEHAB/brevican requires ADAMTS-mediated proteolytic cleavage to promote glioma invasion. J. Neurooncol., 2008.
70. Ruoslahti, E. Brain extracellular matrix. Glycobiology 6, 489, 1996.
71. Novak, U., and Kaye, A. H. Extracellular matrix and the brain: components and function. J. Clin. Neurosci. 7, 280, 2000.
72. Bandtlow, C. E., and Zimmermann, D. R. Proteoglycans in the developing brain: new conceptual insight or old proteins. Physiol Rev 80, 1267, 2000.
73. Morgenstern, D. A., Asher, R. A., and Fawcett, J. W. Chondroitin sulphate proteoglycans in the CNS injury response. Prog Brain Res 137, 313, 2002.
74. Properzi, F., and Fawcett, J. W. Proteoglycans and brain repair. News Physiol Sci. 19, 33, 2004
75. Viapiano, M. S., and Matthews, R. T. From barriers to bridges: chondroitin sulfate proteoglycans in neuropathology. Trends Mol. Med. 12, 488, 2006.
76. Toole, B. P. Hyaluronan and its binding proteins, the hyaladherins. Curr Opin Cell Biol 2, 839, 1990.
77. Yamaguchi, Y. Lecticans: organizers of the brain extracellular matrix. Cell Mol Life Sci 57, 276, 2000.
78. Rauch, U. Extracellular matrix components associated with remodeling processes in brain. Cell Mol. Life Sci. 61, 2031, 2004.
79. Delpech, B., Delpech, A., Bruckner, G., Girard, N., and Maingonnat, C. Hyaluronan and hyaluronectin in the nervous system. Ciba Found Symp 143, 208, 1989.
80. Wu, Y., Wu, J., Lee, D. Y., Yee, A., Cao, L., Zhang, Y., Kiani, C., and Yang, B. B. Versican protects cells from oxidative stress-induced apoptosis. Matrix Biol. 24, 3, 2005.
81. Morawski, M., Bruckner, M. K., Riederer, P., Bruckner, G., and Arendt, T. Perineuronal nets potentially protect against oxidative stress. Exp. Neurol. 188, 309, 2004.
82. Albertini, R., Passi, A., Abuja, P. M., and De, L. G. The effect of glycosaminoglycans and proteoglycans on lipid peroxidation. Int. J. Mol. Med. 6, 129, 2000.
83. Lesma, E., Di Giulio, A. M., Ferro, L., Prino, G., and Gorio, A. Glycosaminoglycans in nerve injury: 1. Low doses of glycosaminoglycans promote neurite formation. J. Neurosci. Res. 46, 565, 1996.
84. Silver, J., and Miller, J. H. Regeneration beyond the glial scar. Nat. Rev. Neurosci. 5, 146, 2004.
85. Rao, J. S. Molecular mechanisms of glioma invasiveness: the role of proteases. Nat. Rev. Cancer 3, 489, 2003.
86. Binder, D. K., and Berger, M. S. Proteases and the biology of glioma invasion. J. Neurooncol. 56, 149, 2002.
87. Gladson, C. L. The extracellular matrix of gliomas: Modulation of cell function. Journal of Neuropathology and Experimental Neurology 58, 1029, 1999.
88. Cattaruzza, S., and Perris, R. Proteoglycan control of cell movement during wound healing and cancer spreading. Matrix Biol. 24, 400, 2005.
89. Goldbrunner, R. H., Bernstein, J. J., and Tonn, J. C. Cell-extracellular matrix interaction in glioma invasion. Acta Neurochir (Wien) 141, 295, 1999.
90. Aboitiz, F., Scheibel, A. B., Fisher, R. S., and Zaidel, E. Fiber composition of the human corpus callosum. Brain Res. 598, 143, 1992.
91. Farin, A., Suzuki, S. O., Weiker, M., Goldman, J. E., Bruce, J. N., and Canoll, P. Transplanted glioma cells migrate and proliferate on host brain vasculature: a dynamic analysis. Glia 53, 799, 2006.
92. Farin, A., Suzuki, S. O., Weiker, M., Goldman, J. E., Bruce, J. N., and Canoll, P. Transplanted glioma cells migrate and proliferate on host brain vasculature: a dynamic analysis. Glia 53, 799, 2006.
93. Giese, A., Loo, M. A., Rief, M. D., Tran, N., and Berens, M. E. Substrates for astrocytoma invasion. Neurosurgery 37, 294, 1995.
94. Xie, F., and Zheng, B. White matter inhibitors in CNS axon regeneration failure. Exp. Neurol. 209, 302, 2008.
95. Giese, A., Laube, B., Zapf, S., Mangold, U., and Westphal, M. Glioma cell adhesion and migration on human brain sections. Anticancer Res. 18, 2435, 1998.
96. Giese, A., Loo, M. A., Tran, N., Haskett, D., Coons, S. W., and Berens, M. E. Dichotomy of astrocytoma migration and proliferation. International Journal of Cancer 67, 275, 1996.
97. Xie, F., and Zheng, B. White matter inhibitors in CNS axon regeneration failure. Exp. Neurol. 209, 302, 2008.
98. Davies, S. J., and Silver, J. Adult axon regeneration in adult CNS white matter. Trends Neurosci. 21, 515, 1998.
99. Hu, F., and Strittmatter, S. M. Regulating axon growth within the postnatal central nervous system. Semin.Perinatol. 28, 371, 2004.
100. Grandpre, T., and Strittmatter, S. M. Nogo: a molecular determinant of axonal growth and regeneration. Neuroscientist. 7, 377, 2001.
101. Giese, A., Kluwe, L., Laube, B., Meissner, H., Berens, M. E., and Westphal, M. Migration of human glioma cells on myelin. Neurosurgery 38, 755, 1996.
102. Choi, H. W., Johnson, J. K., Nam, J., Farson, D. F., and Lannutti, J. J. Structuring electrospun polycaprolactone nanofiber tissue scaffolds by femtosecond laser ablation. Journal of Laser Applications 19, 225, 2007.
103. Chalmers, J. J., Tong, X., Lara, O., and Moore, L. R. Preparative applications of magnetic separation in biology and medicine. In: Zborowski, M., and Chalmers, J. J., eds. Laboratory Techniques in Biochemistry and Molecular Biology, 2007, pp. 249-264.
104. Zborowski, M., Ostera, G. R., Moore, L. R., Milliron, S., Chalmers, J. J., and Schechter, A. N. Red blood cell magnetophoresis. Biophysical Journal 84, 2638, 2003.
105. Mehta, B., Yang, L., and Chalmers, J. J. Optimization of enzyme dissociation protocol to digest tumor tissue based on a reaction diffusion model. Biotechnology and Bioengineering submitted, 2008.
106. Tomlinson, I. P. M., Lambros, M. B. K., and Roylance, R. R. Loss of heterozygosity analysis: Practically and conceptually flawed? Genes Chromosomes & Cancer 34, 349, 2002.
107. Sieben, N. L. G., ter Haar, N. T., Cornelisse, C. J., Fleuren, G. J., and Cleton-Jansen, A. M. PCR artifacts in LOH and MSI analysis of microdissected tumor cells. Human Pathology 31, 1414, 2000.
108. Ko, Y., Grunewald, E., Totzke, G., Klinz, M., Fronhoffs, S., Gouni-Berthold, I., Sachinidis, A., and Vetter, H. High percentage of false-positive results of cytokeratin 19 RT-PCR in blood: A model for the analysis of illegitimate gene expression. Oncology 59, 81, 2000.
109. Hu, B., Kong, L., Matthews, R. T., and Viapiano, M. S. The proteoglycan brevican binds to fibronectin after proteolytic cleavage and promotes glioma cell adhesion and motility. Journal of Biologica Chemistry (submitted), 2008.
110. Williams, S., Nowicki, M., Godlewski, J., Chiocca, E. A., and Lawler, S. Anti-glioma effects of protein kinase inhibitors that simultaneously block invasion and proliferation. Neuro-Oncology 9, 486, 2007.
111. Stein, A., Nowicki, M., Lawler, S., Chiocca, E., Demuth, T., Berens, M., and Sander, L. Estimating the cell density and invasive radius of 3d glioblastoma tumor spheroids grown in vitro. Applied Optics in press, 2007.
112. Camoretti-Mercado, B., Targeting the airway smooth muscle for asthma treatment. Translational Research, 2009. 154(4): p. 165-174.
113. Barth, P. J. and C. C. Westhoff, CD34+ fibrocytes: morphology, histogenesis and function. Curr. Stem Cell. Res. Ther., 2007. 2(3): p. 221-227.
114. Schmidt, M., et al., Identification of circulating fibrocytes as precursors of bronchial myofibroblasts in asthma. Journal of Immunology, 2003. 171(1): p. 380-389.
115. Bucala, R., et al., CIRCULATING FIBROCYTES DEFINE A NEW LEUKOCYTE SUBPOPULATION THAT MEDIATES TISSUE-REPAIR. Molecular Medicine, 1994. 1(1): p. 71-81.
116. Mori, L., et al., Fibrocytes contribute to the myofibroblast population in wounded skin and originate from the bone marrow. Experimental Cell Research, 2005. 304(1): p. 81-90.
117. Saunders, R., et al., Fibrocyte localization to the airway smooth muscle is a feature of asthma. Journal of Allergy and Clinical Immunology, 2009. 123(2): p. 376-384.
118. Wang, C. H., et al., Increased circulating fibrocytes in asthma with chronic airflow obstruction. American Journal of Respiratory and Critical Care Medicine, 2008. 178(6): p. 583-591.
119. Geiser, M., et al., The role of macrophages in the clearance of inhaled ultrafine titanium dioxide particles. American Journal of Respiratory Cell and Molecular Biology, 2008. 38(3): p. 371-376.
120. Alexis, N. E., et al., In vivo particle uptake by airway macrophages in healthy volunteers. American Journal of Respiratory Cell and Molecular Biology, 2006. 34(3): p. 305-313.
121. Kim, K. K., et al., Epithelial cell alpha 3 beta 1 integrin links beta-catenin and Smad signaling to promote myofibroblast formation and pulmonary fibrosis. Journal of Clinical Investigation, 2009. 119(1): p. 213-224.
122. Murray, L. A., et al., Hyper-responsiveness of IPF/UIP fibroblasts: Interplay between TGF beta 1, IL-13 and CCL2. International Journal of Biochemistry & Cell Biology, 2008. 40(10): p. 2174-2182.
123. Ninomiya, K., et al., Transforming growth factor-beta signaling enhances transdifferentiation of macrophages into smooth muscle-like cells. Hypertension Research, 2006. 29(4): p. 269-276.
124. Epperly, M. W., et al., Correlation of ionizing irradiation-induced late pulmonary fibrosis with longterm bone marrow culture fibroblast progenitor cell biology in mice homozygous deletion recombinan negative for endothelial cell adhesion molecules. In Vivo, 2004. 18(1): p. 1-14.
125. Yang, C. Q., et al., Integrin alpha 1 beta 1 and alpha 2 beta 1 are the key regulators of hepatocarcinoma cell invasion across the fibrotic matrix microenvironment. Cancer Research, 2003. 63(23): p. 8312-8317.
126. Sasmono, R. T., et al., A macrophage colony-stimulating factor receptor-green fluorescent protein transgene is expressed throughout the mononuclear phagocyte system of the mouse. Blood, 2003. 101(3): p. 1155-1163.
127. Bershadsky, A., M. Kozlov, and B. Geiger, Adhesion-mediated mechanosensitivity: a time to experiment, and a time to theorize. Current Opinion in Cell Biology, 2006. 18(5): p. 472-481.
128. Discher, D. E., P. Janmey, and Y. L. Wang, Tissue cells feel and respond to the stiffness of their substrate. Science, 2005. 310(5751): p. 1139-1143.
129. Hinz, B., et al., Alpha-smooth muscle actin expression upregulates fibroblast contractile activity. Molecular Biology of the Cell, 2001. 12(9): p. 2730-2741.
130. Cukierman, E., et al., Taking cell-matrix adhesions to the third dimension. Science, 2001. 294(5547): p. 1708-1712.
131. Beningo, K. A., et al., Nascent focal adhesions are responsible for the generation of strong propulsive forces in migrating fibroblasts. Journal of Cell Biology, 2001. 153(4): p. 881-887.
132. Pelham, R. J. a. W., Y.-L., Cell locomotion and focal adhesions are regulated by substrate flexibility. Proceedings of the National Academy of Science USA, 1997. 94: p. 13661-13665.
133. Engler, A. J., et al., Matrix elasticity directs stem cell lineage specification. Cell, 2006. 126(4): p. 677- 689.
134. Liu S L, Sugimoto Y, Sorio C, Tecchio C, Lin Y C 2004 Function analysis of estrogenically regulatedprotein tyrosine phosphatase gamma (PTP gamma) in human breast cancer cell line MCF-7. Oncogene 23(6):1256-1262.
135. Liu S L, Kulp S K, Sugimoto Y, Jiang J H, Chang H L, Lin Y C 2002 Involvement of breast epithelialstromal interactions in the regulation of protein tyrosine phosphatase-gamma (PTP gamma) mRNA expression by estrogenically active agents. Breast Cancer Research and Treatment 71(1):21-35.
136. Liu S, Lin Y C 2004 Transformation of MCF-10A human breast epithelial cells by zeranol and estradiol-17beta. Breast 10(6):514-521.
137. Wang L S, Huang Y W, Sugimoto Y, Liu S L, Chang H L, Ye W P, Shu S, Lin Y C 2006 Conjugated linoleic acid (CLA) up-regulates the estrogenregulated cancer suppressor gene, protein tyrosine phosphatase gamma (PTP gamma), in human breast cells. Anticancer Research 26(1A):27-34.
138. Nam, J., Huang, Y., Agarwal, S., and Lannutti, J. Materials Selection and Residual Solvent Retention in Biodegradable Electrospun Fibers. Journal Of Applied Polymer Science 107, 1547, 2008.
139. Johnson, J., Niehaus, A., Nichols, S., Lee, D., Koepsel, J., Anderson, D., and Lannutti, J. Electrospun PCL In Vitro: A Microstructural Basis for Mechanical Property Changes. Journal of Biomaterials Science Polymer Edition in press, 2008.
140. Duling, R. R., Dupaix, R. B., Katsube, N., and Lannutti, J. Mechanical characterization of electrospun polycaprolactone (PCL): a potential scaffold for tissue engineering. Journal of Biomechanical Engineering 130 2008.
141. Drilling, S., Gaumer, J., and Lannutti, J. Burst Pressure Competent Vascular Grafts via Electrospinning: Microstructural Control. Journal of Biomedical Materials Research in press, DOI #10.1002/jbm.a.31926, 2008.
142. Nam, J., Huang, Y., Agarwal, S., and Lannutti, J. Improved Cellular Infiltration in Electrospun Fiber via Engineered Porosity. Tissue Engineering 13, 22249, 2007.
143. Huang, Z. M., Zhang, Y. Z., Kotaki, M., and Ramakrishna, S. A review on polymer nanofibers by electrospinning and their applications in nanocomposites. Composites Science and Technology 63, 2223, 2003.
144. Katta, P., Alessandro, M., Ramsier, R. D., and Chase, G. G. Continuous electrospinning of aligned polymer nanofibers onto a wire drum collector. Nano Letters 4, 2215, 2004.
145. Li, D., Wang, Y. L., and Xia, Y. N. Electrospinning nanofibers as uniaxially aligned arrays and layer-bylayer stacked films. Advanced Materials 16, 361, 2004.
146. Li, D., and Xia, Y. N. Electrospinning of nanofibers: Reinventing the wheel? Advanced Materials 16, 1151, 2004.
147. Teo, W. E., and Ramakrishna, S. Electrospun fibre bundle made of aligned nanofibres over two fixed points. Nanotechnology 16, 1878, 2005.
148. Yu, J. H., Fridrikh, S. V., and Rutledge, G. C. Production of submicrometer diameter fibers by two-fluid electrospinning. Adv. Mater. 16, 1562, 2004.
149. Swanson, K. R., Alvord, E. C., and Murray, J. D. A quantitative model for differential motility of gliomas in grey and white matter. Cell Proliferation 33, 317, 2000.
150. Swanson, K. R. Quantifying glioma cell growth and invasion in vitro.

Mathematical and Computer Modelling 47, 638, 2008.
151. Chicoine, M. R., and Silbergeld, D. L. Assessment of brain-tumor cell motility in-vivo and in-vitro. Journal of Neurosurgery 82, 615, 1995.
152. Norton, W. T., and Poduslo, S. E. Myelination in rat brain: method of myelin isolation. J. Neurochem. 21, 749, 1973.
153. Haley, J. E., Samuels, F. G., and Ledeen, R. W. Study of myelin purity in relation to axonal contaminants. Cell Mol. Neurobiol. 1, 175, 1981.
154 Lannutti, J., et al., Electrospinning for tissue engineering scaffolds. Materials

Science and Engineering: C 2007. 27: p. 504-509.
155. Johnson, J., et al., Electrospun PCL in Vitro: a Microstructural Basis for Mechanical Property Changes. Journal of Biomaterials Science-Polymer Edition, 2009. 20(4): p. 467-481.
156. Gaumer, J., et al., Source-to-ground Distance and the Mechanical Properties of Electrospun Fiber. Acta Biomaterialia, 2009. 5(5): p. 1552-1561.
157. Nam, J., et al., Materials Selection and Residual Solvent Retention in Biodegradable Electrospun Fibers. Journal Of Applied Polymer Science, 2008. 107(3): p. 1547-1554.
158. Baran, C. P., et al., Important roles for macrophage colony-stimulating factor, CC chemokine ligand 2, and mononuclear phagocytes in the pathogenesis of pulmonary fibrosis. American Journal of Respiratory and Critical Care Medicine, 2007. 176(1): p. 78-89.
159. Powell, H. M. and S. T. Boyce, EDC cross-linking improves skin substitute strength and stability. Biomaterials, 2006. 27(34): p. 5821-5827.
160. Srikar, R., et al., Desorption-limited mechanism of release from polymer nanofibers. Langmuir, 2008. 24(3): p. 965-974.
161. Xie, J. W., et al., Encapsulation of protein drugs in biodegradable microparticles by co-axial electrospray. Journal of Colloid and Interface Science, 2008. 317(2): p. 469-476.
162. Kim, T. G., D. S. Lee, and T. G. Park, Controlled protein release from electrospun biodegradable fiber mesh composed of poly(epsilon-caprolactone) and poly(ethylene oxide). International Journal of Pharmaceutics, 2007. 338(1-2): p. 276-283.
163. Diaz, J. E., et al., Controlled encapsulation of hydrophobic liquids in hydrophilic polymer nanofibers b co-electrospinning. Advanced Functional Materials, 2006. 16(16): p. 2110-2116.
164. Nam, J., J. Lannutti, and S. Agarawal, manuscript in preparation. 2010.

We claim:

1. A culture cell for growing animal cells in vitro, which comprises:
a cell having sides and a bottom forming a volume, said volume containing a layer of oriented nanofiber upon which animal cells can be cultured.

2. The culture cell of claim 1, wherein at least two layers of nanofiber are contained in said volume, at least one of said layers being oriented nanofiber.

3. The culture cell of claim 2, wherein said layers are formed of nanofiber having one or more of different morphology, porosity, density, electrical conductivity, or stiffness.

4. The culture cell of claim 3, wherein said layers are formed of nanofiber having different composition.

5. The culture cell of claim 1, an outer surface of said oriented nanofiber is subjected to treatment.

6. The culture cell of claim 5, wherein said treatment is one or more of a coating applied to said outer surface; said outer surface subjected to super-critical CO₂ treatment; said outer surface subjected to sub-critical CO₂ treatment; said outer surface subjected to a chemotactic source; or said outer surface subjected to treatment to form one or more of pores, dimples, grass, or hair.

7. The culture cell of claim 6, wherein said oriented nanofiber is coated with one or more of cells or biological milieu derived from homogenized whole organs, organ-derived fluids or matrices surrounding specific cells associated with either (a) an organ of interest or (b) a disease of interest.

8. The culture cell of claim 1, wherein said oriented nanofiber has a core/shell construction.

9. The culture cell of claim 1, wherein said oriented nanofiber has a linear density ranging from between about 1 fiber/mm to about 200,000 fibers/mm.

10. A method for culturing animal cells in vitro, which comprises the steps of:
(a) in a cell having sides and a bottom forming a volume, placing a layer of oriented nanofiber in said volume;
(b) inoculating said oriented nanofiber layer with animal cells; and
(c) establishing cell culture conditions in said cell for culturing said animal cells.

11. The method of claim 10, wherein at least two layers of nanofiber are contained in said volume, at least one of said layers being oriented nanofiber.

12. The method of claim 11, wherein said layers are formed of nanofiber having one or more of different morphology, porosity, density, electrical conductivity, or stiffness.

13. The method of claim 12, wherein said layers are formed of nanofiber having different composition.

14. The method of claim 10, an outer surface of said oriented nanofiber is subjected to treatment.

15. The method of claim 14, wherein said treatment is one or more of a coating applied to said outer surface; said outer surface subjected to super-critical CO₂ treatment; said outer surface subjected to sub-critical CO₂ treatment; said outer surface subjected to a chemotactic source; or said outer surface subjected to treatment to form one or more of pores, dimples, grass, or hair.

16. The method of claim 14,wherein said oriented nanofiber is coated with one or more of cells or biological milieu derived from homogenized whole organs, organ-derived fluids or matrices surrounding specific cells associated with either (a) an organ of interest or (b) a disease of interest.

17. The method of claim 10, wherein said oriented nanofiber has a core/shell construction.

18. The method of claim 10, wherein said oriented nanofiber has a linear density ranging from between about 1 fiber/mm to about 200,000 fibers/mm.

19. The method of claim 10, one or more types of cells are inoculated and at least two genetically different cell types are separated based on cell motility along the oriented nanofiber.

20. The method of claim 10, which additional comprises the step of in vitro evaluation of one or more of cosmetic products, cell radiation exposure, chemotherapeutics, dietary influences on cell development, cell-cell communication, anti-migratory compounds, cell separations, or oxygen tension effects.

21. A method of making a culture cell for growing animal cells in vitro, which comprises the steps of:
(a) providing an array of culture cells having sides and a bottom forming a volume, wherein electrical ground is established between each said culture cell in said array; and
(b) electrospinning a layer of oriented nanofiber onto said array.

22. The method of claim 21, wherein material being subject to said electrospinning is dispersed in one or more of hexafluoroisoproponal (HFIP or HFP), acetone, dichloromethane, trifluoroacetic acid, acetic acid, petroleum either, or dimethylformamide.

23. A method of making oriented nanofiber for use in a culture cell for growing animal cells in vitro, which comprises the steps of:
(a) electrospinning a layer of oriented nanofiber onto a moving surface to form oriented nanofiber; and
(b) collecting said oriented nanofiber from said surface.

24. The method of claim 23, wherein said collected oriented nanofiber is cut to length.

25. The method of claim 24, wherein a bottomless well plate is glued onto said cut oriented nanofiber.

26. The method of claim 23, wherein material being subject to said electrospinning is dispersed in one or more of hexafluoroisoproponal (HFIP or HFP), acetone, dichloromethane, trifluoroacetic acid, acetic acid, petroleum either, or dimethylformamide.
